(19) United States
(12) Patent Application Publication
(10) Pub. No.: US 2008/0274543 A1
(43) Pub. Date: Nov. 6, 2008

(54) Neuronal Cell Propagation Using Rotating Wall Vessel

(75) Inventors: Mario T. Philipp, Mandeville, LA (US); Cheryl A. Nickerson, Phoenix, AZ (US); Tereance A. Myers, Covington, LA (US)

Correspondence Address: CHESTER G. MOORE;Baker, Donelson, Bearman, Caldwell & Berkowitz, PC, 3 Sanctuary Boulevard, SUITE 201, Mandeville, LA, 70471, US

(73) Assignee: THE ADMINISTRATORS OF THE TULANE EDUCATIONAL FUND, New Orleans, LA (US)

(21) Appl. No.: 12/113,667

(22) Filed: May 1, 2008

Related U.S. Application Data
(60) Provisional application No. 60/915,407, filed on May 1, 2007.

(51) Int. Cl. C12N 5/06; C12N 5/08

(52) U.S. Cl. 435/353; 435/394; 435/325; 435/368

(57) ABSTRACT

The present invention provides methods of propagating transformed neurons in a simulated microgravity environment generated by a rotating wall vessel (“3-D culture”) so that the phenotype of the transformed neurons so cultured becomes closer to that of non-transformed neurons (primary neurons) and less like the phenotype of transformed neurons cultured via standard cell culture techniques (“2-D culture”).

### CROSS-REFERENCE TO RELATED APPLICATIONS

This Non-Provisional Patent Application, filed under 35 U.S.C. § 111 (a), claims the benefit under 35 U.S.C. § 119(e)(1) of U.S. Provisional Patent Application No. 60/915,407, filed under 35 U.S.C. § 111 (b) on 1 May 2007, and which is hereby incorporated by reference in its entirety.

### STATEMENT REGARDING FEDERALLY SPONSORED RESEARCH

The invention was made with U.S. Government support under grant numbers NS048952 and RR00164 (MTP) awarded by the National Institutes of Health. The United States Government has certain rights in the invention.

### THE NAMES OF THE PARTIES TO A JOINT RESEARCH AGREEMENT

Not applicable.

### INCORPORATION-BY-REFERENCE OF MATERIAL SUBMITTED ON COMPACT DISC

Not applicable.

### BACKGROUND OF THE INVENTION

1. Field of the Invention

The present invention relates to methods of culturing neurons for in vitro laboratory investigations. More particularly, the present invention relates to methods of culturing transformed neurons in 3-D culture so that their phenotype (“3-D phenotype”) becomes more like the phenotype of non-transformed neurons (primary neurons) and less like the phenotype of transformed neurons in 2-D culture (“2-D phenotype”).

2. Description of Related Art

Neurons, also known as neurones, neuronal cells, or nerve cells, are the primary functional units of the central nervous system. They comprise the core components of the brain, spinal cord, and peripheral nerves. Neurons are electrically excitable cells that process and transmit information via chemical and electrical synapses through a process known as synaptic transmission. Synaptic transmission is triggered by the action potential, a propagating electrical signal that is generated by exploiting the electrically excitable membrane of the neuron.

Neurons are typically composed of a cell body, called a soma, a dendritic tree (branched projections of a neuron that act to conduct the electrical stimulation received from other neural cells to soma), and an axon, which is a nerve fiber that conducts electrical impulses away from the soma.

Neurons display a diversity of structures and functions and are classified accordingly. Structurally, neurons are grouped according to their anatomical shape or their location in the nervous system. Unipolar or pseudipolar neurons have a dendrite and axon emerging from the same process while bipolar neurons have a single axon and single dendrite on opposite ends of the soma. Multipolar neurons have more than two dendrites and are sub-classified as Golgi I (neurons with long-projecting axonal processes) or Golgi II (neurons whose axonal process projects locally). Additional shape and location classifications of neurons include Basket, Betz, medium spiny, Purkinje, pyramidal, and Renshaw.

Neuronal functional groups include afferent neurons, efferent neurons, and interneurons. Afferent neurons convey information from tissues and organs into the central nervous system (CNS). Efferent neurons, sometimes called motor neurons, transmit signals from the central nervous system to the effector cells (e.g., muscle cells). Interneurons connect neurons to other neurons within specific regions of the central nervous system (e.g., spinal cord). Neurons may be classified by various methods, including: according to their action on other neurons (e.g., excitatory, inhibitory, etc.); their discharge patterns (i.e., as detected by electrophysiological techniques); neurotransmitter released (e.g., cholinergic, dopaminergic, etc.); and species, tissue source, and developmental stage (e.g., embryonic mouse cerebellar neurons).

Neurological diseases are disorders of the brain, spinal cord, and nerves; the latter are composed primarily of neurons. There are approximately six hundred known neurological diseases, which can be caused by a multitude of factors, including but not limited to faulty genes, nervous system development, degenerative diseases, diseases of the vessels that supply blood to the brain, injuries to the brain and spinal cord, seizure disorders, cancers, chemicals, and infections. Three common neurological diseases include Alzheimer's disease (AD), Huntington's disease (HD), and Parkinson's disease (PD).

Alzheimer's disease is the most common form of dementia, a group of conditions that all gradually destroy neurons and other brain cells and lead to progressive decline in mental function. Vascular dementia, another common form, results from reduced blood flow to the brain's neurons and other nerve cells. In some cases, Alzheimer's disease and vascular dementia can occur together in a condition called “mixed dementia.” Alzheimer's disease is a progressive brain disorder that gradually destroys a person's memory and ability to learn, reason, make judgments, communicate, and carry out daily activities. It is characterized by amyloid plaques (abnormal clumps) and neurofibrillary tangles (abnormal tangles of fibers) within the brain. These plaques and tangles are comprised of aberrant proteins (including amyloid beta). As Alzheimer's disease progresses, individuals may also experience changes in personality and behavior, such as anxiety, suspiciousness or agitation, as well as delusions or hallucinations. The most striking early symptom is loss of short-term memory (amnesia), which usually manifests as minor forgetfulness that becomes steadily more pronounced with illness progression, with relative preservation of older memories. As the disorder progresses, cognitive (intellectual) impairment extends to the domains of language (aphasia), skilled movements (apraxia), recognition (agnosia), and those functions (such as decision-making and planning) closely related to the frontal and temporal lobes of the brain as they become disconnected from the limbic system, reflecting concomitant progression of the underlying pathological processes. These pathological processes consist principally of neuronal loss or atrophy, principally in the temporoparietal cortex, but also in the frontal cortex, together with an inflammatory response to the deposition of amyloid plaques and neurofibrillary tangles. Alzheimer's disease was the seventh leading cause of death in the United States in 2004, claiming an estimated 66,000 lives that year. It is currently the third most costly disease in the United States, after heart disease and cancer. More than five million Americans have been diagnosed with Alzheimer's disease, and that number is expected to increase to eighty-one million by the year 2040. The average lifetime cost of care for a person with Alzheimer's disease is estimated to be $174,000.

Huntington's disease (HD) is the result of the degeneration of neurons in the basal ganglia of the brain. The basal ganglia are structures deep within the brain, involved in many important functions, including coordination of movement. In the basal ganglia, HD affects neurons of the striatum in particular, especially those in the caudate nuclei and the pallidum (globus pallidus). The cerebral cortex, which governs memory, thought, and perception, is also affected in HD. The neurodegeneration associated with HD causes uncontrolled movements, loss of intellectual faculties, and emotional disturbance. It is a familial disease, passed from parent to child through a trinucleotide repeat expansion (a mutation) in the Huntingtin (Htt) gene, and is one of several expanded polyglutamine (PolyQ, or triplet repeat expansion) diseases. This expansion produces a mutant form of the Htt protein (mHtt), which results in neuronal cell death in select areas of the brain, and is a terminal illness. Although Huntington's disease is an inherited disease, there have been rare cases of the disease occurring in individuals with no family history. It affects an estimated 30,000 people in the United States; estimates of its prevalence are about 1 in 10,000 people. Huntington's disease affects an estimated 3 to 7 per 100,000 people of European ancestry.

Parkinson's disease is a disorder that affects neurons and other nerve cells in the part of the brain that controls muscle movement (particularly the dopaminergic neurons of the substantia nigra). The pronounced motor disturbances that are associated with PD are largely the result of degeneration of dopaminergic neurons in the substantia nigra pars compacta, which leads to decreased stimulation of the motor cortex by the basal ganglia (and by the caudate nucleus and putamen in particular). Secondary symptoms may include high-level cognitive dysfunction and subtle language problems. PD is both chronic and progressive. Unlike other serious neurological diseases, Parkinson's is treatable either through medication, implanted devices, or surgery. Nevertheless, the benefits of drug therapy often wane after as little as 5 years of treatment, and the drugs themselves produce undesirable side-effects. As many as one million Americans suffer from Parkinson's disease, which is more than the combined number of people diagnosed with multiple sclerosis, muscular dystrophy and Lou Gehrig's disease. Approximately 40,000 Americans are diagnosed with Parkinson's disease each year, and this number does not reflect the thousands of cases that go undetected. Incidence of Parkinson's increases with age, but an estimated 15 percent of people with PD are diagnosed before the age of 50. The amount of money that the United States and individual patients spend each year on Parkinson's disease is staggering. The combined direct and indirect cost of Parkinson's, including treatment, social security payments, and lost income from inability to work is estimated to be nearly $25 billion per year in the United States alone. Medication costs for an individual patient average $2,500 a year, and therapeutic surgery can cost up to $100,000 dollars per patient.

Alzheimer's Disease, Huntington's Disease and Parkinson's Disease are all relatively poorly understood at this point. The development of successful treatments for these and other neurological diseases would be greatly expedited and facilitated by the availability of human neuronal cell cultures that can be easily propagated and accurately represent, in vitro, the naturally occurring state of neurons in vivo. At present, such accurate and useful human neuronal cell cultures do not exist.

Cell culture is an in vitro tool for studying cell behavior, investigating cellular responses to various stimuli, determining drug efficacy and toxicity ex vivo, and facilitating drug discovery. In vitro studies of disease pathogenesis in the CNS are often conducted with cultures of primary cells, but when the cells in question are neurons—human neurons, in particular—this becomes problematic because most post-embryonic neurons do not divide. Thus, the usefulness of neurons in primary culture is severely limited and researchers must employ transformed neuronal cell lines instead (Encinas M, Iglesias M, Liu Y, Wang H, Muhaisen A, Cena V, Gallego C, Comella J X. Sequential treatment of SH-SY5Y cells with retinoic acid and brain-derived neurotrophic factor gives rise to fully differentiated, neurotrophic factor-dependent, human neuron-like cells. Journal of neurochemistry, 2000; 75: 991-1003; Smith CUM. Elements of Molecular Neurobiology. Second ed. John Wiley and Sons, Ltd: Chichester, 1996). Transformed (or “immortalized”) neuronal cell lines of both human and non-human origin have thus become a requisite tool in studies of neuronal dysfunction in the CNS. While immortalized cell lines are available for most types of non-neuronal mammalian cells, as well as for many specific disease states, there are very few useful neuronal cell lines available for the study of neurological diseases.

The reason behind the limited availability of neuronal cells is that neuronal cells are particularly difficult to culture. They are highly specialized in nature and are extremely selective about the environment in which they grow. Neural tumors usually serve as the principal source of immortalized neural cell lines that are available for biomedical research, in part because they will divide. However, these cell lines are also inherently abnormal since, among other characteristics, they exhibit unregulated cellular division, are known to exhibit an arrested state of cellular differentiation (Abbott A. Cell culture: biology's new dimension. Nature, 2003; 424: 870-2; Guidi A, Dubini G, Tominetti F, Raimondi M. Mechanobiologic Research in a Microgravity Environment Bioreactor. 2002: 1-9; Hanada M, Krajewski S, Tanaka S, Cazals-Hatem D, Spengler B A, Ross R A, Biedler J L, Reed J C. Regulation of Bcl-2 oncoprotein levels with differentiation of human neuroblastoma cells. Cancer research, 1993; 53: 4978-86; van Golen C M, Soules M E, Grauman A R, Feldman E L. N-Myc overexpression leads to decreased beta1 integrin expression and increased apoptosis in human neuroblastoma cells. Oncogene, 2003; 22: 2664-73; Zhang S. Beyond the Petri dish. Nature biotechnology, 2004; 22: 151-2), expression of the proto-oncogene N-myc is typically elevated, and resistance to apoptosis is increased. The inherently abnormal phenotypes of neuronal cell lines complicates the interpretation of experimental results derived from these cells when comparing them to non-transformed cells (i.e., neurons from primary cultures) (Fan L, Iyer J, Zhu S, Frick K K, Wada R K, Eskenazi A E, Berg P E, Ikegaki N, Kennett R H, Frantz C N. Inhibition of N-myc expression and induction of apoptosis by iron chelation in human neuroblastoma cells. Cancer research, 2001; 61: 1073-9; Kang J H, Rychahou P G, Ishola T A, Qiao J, Evers B M, Chung D H. MYCN silencing induces differentiation and apoptosis in human neuroblastoma cells. Biochemical and biophysical research communications, 2006; 351: 192-7; Smith A G, Popov N, Imreh M, Axelson H, Henriksson M. Expression and DNA-binding activity of MYCN/Max and Mnt/Max during induced differentiation of human neuroblastoma cells. Journal of cellular biochemistry, 2004; 92: 1282-95; van Golen et al., 2003; van Noesel M M, Pieters R, Voute P A, Versteeg R. The N-myc paradox: N-myc overexpression in neuroblastomas is associated with sensitivity as well as resistance to apoptosis. Cancer letters, 2003; 197: 165-72). Thus, the optimal methodology for growing neuronal cell cultures useful in biomedical research has become the focus of several areas of cutting-edge research.

In addition to the limitations introduced by transformed cell lines, traditional monolayer or “2-D” culture systems in Petri dishes are often themselves inadequate to realistically model in vivo conditions (Lelkes P I, Galvan D L, Hayman G T, Goodwin T J, Chatman D Y, Cherian S, Garcia R M, Unsworth B R. Simulated microgravity conditions enhance differentiation of cultured PC12 cells towards the neuroendocrine phenotype. In vitro cellular & developmental biology, 1998; 34: 316-25; Nickerson C A, Goodwin T J, Terlonge J, Ott C M, Buchanan K I, Uicker W C, Emami K, LeBlanc C L, Ramamurthy R, Clarke M S, Vanderburg C R, Hammond T, Pierson D L. Three-dimensional tissue assemblies: novel models for the study of Salmonella enterica serovar Typhimurium pathogenesis. Infection and immunity, 2001; 69: 7106-20; O'Brien L E, Zegers M M, Mostov K E. Opinion: Building epithelial architecture: insights from three-dimensional culture models. Nature reviews, 2002; 3: 531-7; Zhang, 2004). Gravity-induced sedimentation, non-homologous delivery of nutrients, and a lack of cell-cell and cell-extracellular matrix contacts are all potential limitations of 2-D cell culture (Abbott, 2003; Guidi et al., 2002; LaMarca H L, Ott C M, Honer Zu Bentrup K, Leblanc C L, Pierson D L, Nelson A B, Scandurro A B, Whitley G S, Nickerson C A, Morris C A. Three-dimensional growth of extravillous cytotrophoblasts promotes differentiation and invasion. Placenta, 2005; 26: 709-20; Nickerson et al., 2001). Perhaps more importantly, 2-D cell culture approaches are known to alter gene expression, hinder cellular differentiation, and prevent formation of the complex three-dimensional cellular architecture commonly found in intact tissues and organs (Abbott, 2003; Eisenstein M. Thinking Outside the Dish. Nature Methods, 2006; 3: 1035-43; Freshney R I. Culture of Animal Cells; A Manual of Basic Technique. Wiley-Liss, Inc.: New York, 2000; Honer zu Bentrup K, Ramamurthy R, Ott C M, Emami K, Nelman-Gonzalez M, Wilson J W, Richter E G, Goodwin T J, Alexander J S, Pierson D L, Pellis N, Buchanan K L, Nickerson C A. Three-dimensional organotypic models of human colonic epithelium to study the early stages of enteric salmonellosis. Microbes and infection/Institut Pasteur, 2006; 8: 1813-25; Nickerson et al., 2001; Schmeichel K L, Bissell M J. Modeling tissue-specific signaling and organ function in three dimensions. Journal of cell science, 2003; 116: 2377-88; Zhang, 2004).

While matrigel, collagen, peptide and synthetic nanofiber scaffolds are each being used and developed as more realistic procedures for in vitro cell culture (Abbott, 2003; O'Brien et al., 2002; Schmeichel and Bissell, 2003; Zhang, 2004), NASA-engineered rotating wall vessels (RWV) are also being employed to establish a fluid suspension culture that is capable of inducing biologically meaningful three-dimensional (or “3-D”) growth in vitro (Gao H, Ayyaswamy P S, Ducheyne P. Dynamics of a microcarrier particle in the simulated microgravity environment of a rotating-wall vessel. Microgravity science and technology, 1997; 10: 154-65; Guidi et al., 2002; LaMarca et al., 2005; Nickerson C A, Ott C M. A New Dimension in Modeling Infectious Disease. ASM News, 2004: 169-75). During culture in a RWV, individual cells aggregate into 3-D tissue-like assemblies, developing enhanced states of differentiation and cross communication through cell-cell contacts. Gas exchange and nutrient delivery are optimized under these conditions (Guidi et al., 2002; Nickerson et al., 2001), and the cellular phenotypes, as compared to their 2-D cultured counterparts, become functionally and morphologically more similar to those observed in the parental tissues and organs they represent (Hammond T G, Hammond J M. Optimized suspension culture: the rotating-wall vessel. American journal of physiology, 2001; 281: F12-25; Lelkes et al., 1998; Nickerson and Ott, 2004; Nickerson C A, Richter E G, Ott C M. Studying host-pathogen interactions in 3-D: organotypic models for infectious disease and drug development. J Neuroimmune Pharmacol, 2007; 2: 26-31; Unsworth B R, Lelkes P I. Growing tissues in microgravity. Nature medicine, 1998; 4: 901-7; Zhang, 2004).

The transformed neuronal cell line SH-SY5Y (“SY”) is a third-generation neuroblastoma (an extracranial solid cancer). It is an adrenergic “n” type clone of the “mixed cell” human neuroblastoma line SK-N-SH, and has been used extensively in standard 2-D cultures to study neuronal function, growth, damage in response to insult, degeneration and differentiation (Biedler J L, Helson L, Spengler B A. Morphology and growth, tumorigenicity, and cytogenetics of human neuroblastoma cells in continuous culture. Cancer research, 1973; 33: 2643-52; Garcia-Gil M, Pesi R, Perna S, Allegrini S, Giannecchini M, Camici M, Tozzi M G. 5′-aminoimidazole-4-carboxamide riboside induces apoptosis in human neuroblastoma cells. Neuroscience, 2003; 117: 811-20; Ho R, Minturn J E, Hishiki T, Zhao H, Wang Q, Cnaan A, Maris J, Evans A E, Brodeur G M. Proliferation of human neuroblastomas mediated by the epidermal growth factor receptor. Cancer research, 2005; 65: 9868-75; Martinez T, Pascual A. Identification of genes differentially expressed in SH-SY5Y neuroblastoma cells exposed to the prion peptide 106-126. The European journal of neuroscience, 2007; 26: 51-9; Ribas J, Boix J. Cell differentiation, caspase inhibition, and macromolecular synthesis blockage, but not BCL-2 or BCL-XL proteins, protect SH-SY5Y cells from apoptosis triggered by two CDK inhibitory drugs. Experimental cell research, 2004; 295: 9-24).

An oncogene is a modified gene or a set of nucleotides that code for a protein that increases the malignancy of a tumor cell (i.e., it encodes a protein that is able to transform cells in culture, or produce cancer in animals). A proto-oncogene is the normal cellular gene from which an oncogene arises. N-Myc is a proto-oncogene that is overexpressed in a wide range of human neuronal cancers. When it is specifically mutated or overexpressed, it increases cell proliferation and functions as an oncogene. HuD is a neuronal-specific RNA-binding protein that is a potential regulator of N-Myc expression in human neuroblastoma cells. Whether HuD regulates N-Myc expression and thereby influences tumor aggressiveness is of major interest. The Bcl-2 gene is the prototype for a family of mammalian genes and the proteins they produce. These proteins govern mitochondrial outer membrane permeabilization and have recognized roles in apoptosis. Also called “programmed cell death,” apoptosis is an organized and well-defined mechanism for the demise of cells, and stands in contrast to “necrosis,” or cell death by tissue damage. Interestingly, these proteins can either be pro-apoptotic (e.g., BAX, BAK, and BOK) or anti-apoptotic (e.g., Bcl-2, Bcl-XL).

In 2006, researchers at the National Institute of Standards and Technology developed neuronal cell cultures by maintaining a stock of neuronal precursor cells that continue to divide prior to differentiation but that could be differentiated to produce stable neural cell cultures. Specifically, they applied this methodology to the embryonic carcinoma (P19) cell line. Although they are rapidly-dividing, P19 cells can be induced to differentiate terminally along central nervous system (CNS), skeletal muscle, or cardiac muscle pathways. Using Polyelectrolyte Multilayers (PEMs), which have been used successfully to control cellular attachment to various surfaces, the authors facilitate Neuron-like Cell (NLC) cultures by enabling direct attachment to NLC cell bodies to the surface and neuronal projections across the PEM-treated surfaces. The authors achieved surface patterning by using microfluidic networks to micropattern the PEMs onto poly(dimethylsiloxane) (PDMS), resulting in confined regions of cellular attachment and cellular outgrowth.

Researchers at Northwestern University were able to develop neuronal cell cultures by employing nanofiber networks. Neural progenitor cells were encapsulated in vitro within a three-dimensional network of nanofibers formed by self-assembly of peptide amphiphile molecules. The self-assembly is triggered by mixing cell suspensions in media with dilute aqueous solutions of the molecules, and cells survive the growth of the nanofibers around them. These nanofibers were designed to present to cells the neurite-promoting laminin epitope IKVAV at nearly van der Waals density. Relative to laminin or soluble peptide, the artificial nanofiber scaffold induced very rapid differentiation of cells into neurons, while discouraging the development of astrocytes, star-shaped glial cells that support the growth of neurons. This rapid selective differentiation is linked to the amplification of bioactive epitope presentation to cells by the nanofibers.

There is an ongoing need for improved methods of propagating neuronal cell cultures for use with in vitro laboratory research that may ultimately lead to novel and effective treatments for neurological disorders. The present invention meets this need by providing novel methods of propagating neuronal cell cultures that do not exhibit the shortcomings of cell cultures developed by any of the existing methods.

### BRIEF SUMMARY OF THE INVENTION

The present invention relates to methods of propagating neuronal cell cultures by use of a simulated microgravity environment generated by a rotating wall vessel.

The present invention overcomes inherent limitations of 2-D primary neuronal culture and 2-D culture of transformed neurons in vitro by providing methods of 3-D in vitro neuronal culture that attenuate the phenotypic differences existing between transformed and untransformed neurons. By culturing SY cells under the gentle, low-shear conditions in a RWV, a cell line that expresses classic morphological and functional patterns of neuronal differentiation is obtained.

In one embodiment of the invention is provided a method of culturing neurons, comprising: a) isolating transformed neuronal cells; and culturing said transformed neuronal cells in 3-D culture, said 3-D culture comprising a rotating wall vessel containing said transformed neuronal cells, culture media, and a cell culture matrix, wherein said rotating wall vessel gravity is balanced by oppositely directed physical forces, and so generating 3-D cultured cells, whereby the 3-D cultured cells adopt a 3-D phenotype, and wherein said 3-D phenotype persists for up to 5 days after said 3-D cultured cells are transferred to 2-D culture. In a preferred aspect of this embodiment, the 3-D phenotype comprises decreased N-myc expression. In another preferred aspect of this embodiment, the 3-D phenotype comprises decreased HuD expression. In another preferred aspect of this embodiment, the 3-D phenotype comprises decreased Bcl-2 expression. In another preferred aspect of this embodiment, the 3-D phenotype comprises increased Bax expression. In another preferred aspect of this embodiment, the 3-D phenotype comprises increased Bak expression. In another preferred aspect of this embodiment, the 3-D phenotype comprises increased susceptibility to apoptosis. In another preferred aspect of this embodiment, the 3-D phenotype comprises increased neurite outgrowth. In another preferred aspect of this embodiment, the 3-D phenotype comprises decreased doubling rate.

In another embodiment of the present invention is provided a transformed neuronal cell with 3-D phenotype, wherein the 3-D phenotype comprises: reduced doubling rate; increased susceptibility to apoptosis; and increased neurite formation. In a preferred aspect of this embodiment, the 3-D phenotype persists for up to 5 days after said cell is transferred to 2-D culture. In another preferred aspect of this embodiment, the 3-D phenotype further comprises: reduced N-myc expression; reduced HuD expression; reduced Bcl-2 expression; increased Bax expression; and increased Bak expression. In another preferred aspect of this embodiment, the 3-D phenotype further comprising reduced N-myc expression and reduced Bcl-2 expression persists for up to 5 days after said cell is transferred to 2-D culture. In another preferred aspect of this embodiment, the 3-D phenotype further comprising reduced N-myc expression, reduced HuD expression, reduced Bcl-2 expression, increased Bax expression, and increased Bak expression persists for up to 5 days after said cell is transferred to 2-D culture. In a most preferred aspect of this embodiment, the transformed neuronal cell is an SH-SY5Y cell or a PC12 cell.

### BRIEF DESCRIPTION OF THE DRAWINGS

FIG. 1 shows 3-D culture-induced changes in cell division rates and morphology. After 3 weeks in RWV culture, the doubling rate (hatched bars) of SY cells that were transferred back into 2-D culture for 5 days (SY 2-D) dropped from 1×/40 h to 1×/65 hours, as compared with SY cells that remained in 3-D culture (SY 3-D). No change in viability (solid bars) was observed. Data are shown as the mean (n=4)±SD; *=P<0.001.

FIG. 2 shows micrographs of culture-induced changes in cell division rates and morphology. SY cells grown in standard 2-D tissue culture flasks (top row) sediment to the bottom surface and have a flattened morphology. Culture in a RWV (bottom row) promotes 3-D assembly of the individual cells into large tissue-like aggregates. “SEM”=scanning electron micrograph.

FIG. 3 is a Western blot showing decreased expression of N-myc and HuD in 3-D versus 2-D-cultured SY cells. Western blot analysis reveals a progressive decrease in the expression of N-myc and HuD proteins after 2 and 4 weeks in 3-D culture that does not occur during growth in 2-D culture.

FIG. 4 is a series of confocal images showing decreased expression of the N-myc oncogene (top row) and the neuron-specific RNA-binding protein HuD (bottom row) in 3-D (right column) versus 2-D-cultured (left column) SY cells. The 3-D culture was maintained for 4 weeks. The secondary antibody to N-myc and HuD is labeled with Alexa 488. Propidium iodide (PI) was used as the nuclear stain. The scale bar on each image represents 20 μm.

FIG. 5 shows via confocal microscopy that resistance to apoptosis is diminished in 3-D-cultured SY cells. Expression of the anti-apoptotic protein Bcl-2 (top row) in SY cells cultured for 3 weeks in a RWV is diminished. Pro-apoptotic Bax (middle row) and Bak (bottom row) proteins are up-regulated in 3-D culture. The secondary antibody to Bcl-2, Bax and Bak is labeled with Alexa 488. Propidium iodide or To-Pro was used to stain the nuclei. Scale bars on the images are: Bcl-2 20 μm, Bax 23.81 μm, Bak 40 μm.

FIG. 6A and FIG. 6B are Western blots showing that resistance to apoptosis is diminished in SY cells cultured in 3-D. Western analysis of whole-cell lysates collected from SY cells after three weeks in either 2-D or 3-D culture confirms that Bcl-2 expression is down-regulated in 3-D cells (FIG. 6A), and expression of Bax is up-regulated (FIG. 6B).

FIG. 7 shows via TUNEL analysis that resistance to apoptosis is diminished in SY cells cultured in 3-D. The percent (left axis) of TUNEL-positive SY cells in 3-D culture (3-D+TG) increased 4 to 7-fold (right axis) above those cultured in 2-D (2-D+TG) when treated with TG (10 nM) “3-D pre-tx” means 3-D cells from RWV just before transfer to dish; “2-D+0” means 2-D cells, unstimulated; “2-D+TG” means 2-D cells stimulated with TG; “3-D+0” means 3-D cells, unstimulated; “3-D+TG” means 3-D cells removed from RWV to dish, stimulated with TG; “3-D(RWV)+TG” means 3-D cells treated with TG inside of the RWV. Data are shown as the mean (n=3)±SD; *=P<0.01 (except for the 3-DRWV+TG, where n=1). Left axis: actual percent apoptosis; right axis: arbitrary units of fold-change representing the actual apoptosis.

FIG. 8 shows via TUNEL analysis that resistance to apoptosis is diminished in PC-12 cells cultured in 3-D. TUNEL-positive PC12 cells cultured in 3-D (3-D+TG) increased 3-fold above those cultured in 2-D (2-D+TG), when treated with TG (10 nM). “3-D pre-tx” means 3-D cells from RWV just before transfer to dish; “2-D+0” means 2-D cells, unstimulated; “2-D+TG” means 2-D cells stimulated with TG; “3-D+0” means 3-D cells, unstimulated; “3-D+TG” means 3-D cells removed from RWV to dish, stimulated with TG; “3-D(RWV)+TG” means 3-D cells treated with TG inside of the RWV. Data are shown as the mean (n=3)±SD; *=P<0.035. Left axis: actual percent apoptosis; right axis: arbitrary units of fold change representing the actual apoptosis.

FIG. 9 shows that 3-D culture-driven changes in the phenotypic differentiation markers N-myc (top row) and Bcl-2 (bottom row) are still apparent in SY cells 5 days after return to 2-D growth in tissue culture flasks. Ten days after re-introduction to 2-D growth from a 3-D culture environment (right-most panels), marker expression in the cells returned to a level more analogous to those of cells cultured in 2-D (left-most panels). The secondary antibody to N-myc and Bcl-2 is labeled with Alexa 488. Propidium iodide was used as the nuclear stain. The scale bars on the 2-D and 3-D images represent 20 μm, except for the 5 days images, where the bars represent 40 μm.

FIG. 10 shows a comparison of gene expression in 2-D and 3-D-cultured SY cells using microarray analysis. Changes in gene expression due to cell culture conditions affect cellular disease-related pathways (showing the top three pathways out of 63, in order of significance). Selection threshold=P<0.05.

FIG. 11 shows a comparison of gene expression in 2-D and 3-D-cultured SY cells using microarray analysis. The ten canonical pathways most affected in SY cells grown in 3-D rather than 2-D are 1: cell cycle (G1/S checkpoint regulation); 2: cell cycle (G2/M DNA damage checkpoint regulation); 3: p53 signaling; 4: neuregulin signaling; 5: hypoxia signaling in the cardiovascular system; 6: IGF-1 signaling; 7: IL-2 signaling; 8: insulin receptor signaling; 9: FGF signaling; and 10: P13K/AKT signaling. Bar graph=ratio of gene expression in 3-D cultured cells as compared to those grown in 2-D. Line graph represents significance as −log(p-value) with P<0.05.

FIG. 12 is a graphical representation of gene expression pathways involved in G1/S cell cycle progression.

### DETAILED DESCRIPTION OF THE INVENTION

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

As used herein, the term “phenotype” means any observed physical quality of a cell or organism, as determined by both genetic makeup and environmental influences, including but not limited to its morphology, its response to environmental or extracellular variables (e.g., toxins, temperature, nutrients, physical forces including but not limited to gravity, shear stress, centrifugal force, viscosity, and Coriolis force), and the expression of a specific trait based upon genetic and environmental influences.

The present invention encompasses the use of rotating wall vessels to propagate neuronal cell cultures. It has been discovered that the use of rotating wall vessels to propagate neuronal cell cultures produces neuronal cell cultures that more closely resemble untransformed neurons than the neuronal cell cultures produced through previous methods.

Rotating wall vessels, including models with perfusion, are a significant advance in cell culture technique. The rotating wall vessel is a vertically rotated cylindrical cell culture device with a coaxial tubular oxygenator, as originally described in U.S. Pat. No. 5,026,650, “Horizontally rotated cell culture system with a coaxial tubular oxygenator,” awarded to Schwarz et al., and incorporated herein by reference. The rotating wall vessel induces expression of select tissue-specific proteins in diverse cell cultures. Examples of expression of tissue-specific proteins include carcinoembryonic antigen expression in MIP-101 colon carcinoma cells, prostate specific antigen induction in human prostate fibroblasts, through matrix material induction during chondrocyte culture. The quiescent cell culture environment of the rotating wall vessel balances gravity with shear and other forces without obvious mass transfer tradeoff. The rotating wall vessel provides a simulated micro gravity culture environment suitable for co-cultures of diverse cell types, and three-dimensional tissue construct formation.

The generation of purified primary neurons in numbers satisfactory for experimental study is difficult to achieve with animal cells, and is nearly impossible with human cells. Researchers must therefore rely on transformed cell lines for many studies of CNS disease pathogenesis. The present invention provides a 3-D model of neuronal cell culture that overcomes many of the inherent limitations of primary neuronal culture and culture of transformed neuronal cell lines. The application of this invention to human neuronal culture is particularly attractive in view of the post-mitotic constraints of neurons in primary culture. The present invention demonstrates that 3-D culture evokes changes in SY cell morphology, proliferation, apoptosis resistance, and differentiation states in a manner that narrows the phenotypic gap between those cells and their non-transformed (primary culture) counterparts. As studies involving human neuronal pathogenesis remain largely dependent on in vitro cell culture, this approach can be further exploited to create more realistic environments in which to model nerve cell functions and responses.

Rotating wall vessel technology is being used in clinical medical practice by facilitating pancreatic islet implantation. Pancreatic islets are prepared in rotating wall vessels to maintain production and regulation of insulin secretion. The islets are alginate encapsulated to create a non-inflammatory immune haven, and are implanted into the peritoneal cavity of Type I diabetic patients. This implantation of pancreatic islets has maintained normoglycemia for 18 months in diabetic patients, and progressed to Phase III clinical trials. These vessels have also been applied to, for example, mammalian skeletal muscle tissue, cartilage, salivary glands, ovarian tumor cells, and colon crypt cells. Previous studies on shear stress response in endothelial cells, and rotating wall vessel culture have been limited to structural genes. These studies did not address the issue of a process for the production of functional molecules, such as hormones. Shear stress response elements have not previously been demonstrated in epithelial cells, either for structural genes of production of functional molecules.

It is generally accepted that once developing neurons leave the ventricular and sub-ventricular zones of the CNS, they are terminally differentiated and become persistently postmitotic (Herrup K, Neve R, Ackerman S L, Copani A. Divide and die: cell cycle events as triggers of nerve cell death. J Neurosci, 2004; 24: 9232-9; Potter S M. Distributed processing in cultured neuronal networks. Progress in brain research, 2001; 130: 49-62; Zhu X, Raina A K, Smith M A. Cell cycle events in neurons. Proliferation or death? The American journal of pathology, 1999; 155: 327-9). Although some new neurons are generated in the adult brain, neuronal exit from the cell cycle is typically viewed as permanent (Becker E B, Bonni A. Cell cycle regulation of neuronal apoptosis in development and disease. Progress in neurobiology, 2004; 72: 1-25; Ding X L, Husseman J, Tomashevski A, Nochlin D, Jin L W, Vincent I. The cell cycle Cdc25A tyrosine phosphatase is activated in degenerating postmitotic neurons in Alzheimer's disease. The American journal of pathology, 2000; 157: 1983-90; Herrup et al., 2004; Potter, 2001; Zhu et al., 1999). The inability of neurons to divide often complicates research paradigms that require primary neuronal cultures. While a handful of human neuronal cell lines are available to researchers, their transformed phenotype is less than optimal. One such line, the SY cell line, is an adrenergic “n” type clone of the “mixed cell” human neuroblastoma line SK-N-SH and has been used extensively in standard 2-D cultures to study neuronal function, growth, damage in response to insult, degeneration and differentiation (Biedler et al., 1973; Garcia-Gil et al., 2003; Hanada et al., 1993; Ho et al., 2005; Martinez and Pascual, 2007; Ribas and Boix, 2004). The present invention discloses application of a transitional cell culture technique to these neuronal cells that attenuates some of the aberrant features characteristic of transformed neurons.

Loss of cellular differentiation, combined with an unchecked potential to proliferate, has long been a hallmark in the progression of tumorigenesis (Becker and Bonni, 2004; Herrup et al., 2004; Li W, Sanki A, Karim R Z, Thompson J F, Soon Lee C, Zhuang L, McCarthy S W, Scolyer R A. The role of cell cycle regulatory proteins in the pathogenesis of melanoma. Pathology, 2006; 38: 287-301; Park M T, Lee S J. Cell cycle and cancer. Journal of biochemistry and molecular biology, 2003; 36: 60-5). The present invention discloses that the morphology and proliferation characteristics of 3-D-cultivated SY cells align more with a parental, untransformed phenotype (i.e., the phenotype of primary neurons) than with the phenotype of SY cells grown only in 2-D culture. This altered phenotype, observed after cells are cultured according to the 3-D culture methods disclosed herein, is referred to herein as “3-D phenotype.” Because standard cell culture protocols usually involve culturing cells on the flat surfaces of Petri dishes or flat-sided culturing flasks, those methods are referred to as “2-D culture.” Finally, characterization of the 3-D phenotype is with reference to the 2-D phenotype (i.e., description of the 3-D phenotype as comprising reduced N-myc expression means that expression of N-myc in 3-D cultured cells is reduced as compared to expression of N-myc in 2-D cultured cells).

Two classic prognostic markers of tumorigenicity in neuroblastoma-elevated N-myc and HuD expression—were diminished in 3-D as compared to 2-D-cultured SY cells. A decline in the amount of HuD mRNA and protein in various cell lines has been shown to cause a marked reduction in steady-state levels of mature N-myc mRNA and protein (Chagnovich D, Cohn S L. Binding of a 40-kDa protein to the N-myc 3′-untranslated region correlates with enhanced N-myc expression in human neuroblastoma. The Journal of biological chemistry, 1996; 271: 33580-6; Grandinetti K B, Spengler B A, Biedler J L, Ross R A. Loss of one HuD allele on chromosome #1p selects for amplification of the N-myc proto-oncogene in human neuroblastoma cells. Oncogene, 2006; 25: 706-12; Kang et al., 2006; Lazarova D L, Spengler B A, Biedler J L, Ross R A. HuD, a neuronal-specific RNA-binding protein, is a putative regulator of N-myc pre-mRNA processing/stability in malignant human neuroblasts. Oncogene, 1999; 18: 2703-10; Smith et al., 2004; van Golen et al., 2003), thus even small decreases in HuD protein may be contributing, via the effect of HuD protein on N-myc, to increased cellular differentiation in 3-D-cultured SY cells.

2-D Cell Culture and Reagents

Human SY neuroblastoma cells (American Type Tissue Culture Collection ATCC CRL-2266) and PC12 rat pheochromocytoma cells (ATCC CRL-1721) were each seeded into separate T75 flasks with medium renewal every 3-7 days. The culture flasks for PC12 cells were coated with PureCol collagen (Inamed). Cell propagation was performed as per the ATCC product sheet. Nerve growth factor (Sigma) was added to the PC12 medium at 50 ng/2-D. Penicillin (100 units/ml), streptomycin (100 units/ml) and amphotericin B (0.25 μg/ml) (Gibco/Invitrogen) were added to all media. Trypsin(2.5%)/EDTA(0.38 g/L) was used to dislodge the cells, and Trypan Blue™ stain was used to assess cell viability (Gibco/Invitrogen). Samples from the 2-D cultures were harvested at a passage≦20.

3-D Cell Culture and Reagents

Approximately 10⁷ viable 2-D-cultured SY or PC12 cells were dislodged by trypsin and loaded into 50-ml RWVs (Synthecon) containing 200 mg of Cytodex-3™ micro-carrier beads (Amersham Biosciences) suspended in complete growth medium (ATCC product sheet). Entirely filled vessels were then attached to a rotator base (Synthecon) with initial speed typically set at 18-22 RPM. The RPM were adjusted during cultivation to maintain the cell aggregates in suspension. Complete removal of all bubbles was addressed upon initial rotation and daily thereafter. Cell viability assays and medium replacement were performed every 2-5 days. The cells were collected after 2-4 wk (see individual results) of culture. Although minimal changes were noted at 2 wk, significant molecular marker differences were typically found at 3 weeks, with small additional changes at 4 weeks. For efficiency, 3 weeks was used as the standard.

Cell Counting and Cell Proliferation Assays

3-D cultures were removed from the RWV, dislodged from the Cytodex beads by treatment with trypsin/EDTA, and then dissociated from the beads with 40-μm cell strainers (Becton, Dickinson and Company). One million (106) 2-D and 3-D cultured SY cells were independently seeded into 10 ml of complete growth medium in T75 culture dishes and allowed to propagate for 5 days. Cells were them removed from the dish, (trypsin/EDTA), and counted in a BrightLine Hemocytometer.

Morphology: Light and Electron Microscopy

Live cell photographs were imaged with a Sony Cyber Shot digital still camera (DSCF717) attached to a Nikon TMS light microscope. Scanning electron microscopy (SEM) was used to examine changes in the morphology of SY cells as described previously with minor modifications (Nickerson et al., 2001). 2-D cells and 3-D cell aggregates were fixed in 3% glutaraldehyde, 0.5% paraformaldehyde in PBS, pH 7.2, for a minimum of 24 h. The samples were flushed in triplicate with filter-sterilized deionized water to remove salts and then transferred for observation to a Philips XL 30 ESEM (LEI Co.). Chamber pressure was adjusted between 1 and 2 torr to optimize image quality.

Confocal Microscopy

2-D and 3-D cells removed from culture were washed once in PBS and fixed in 2% paraformaldehyde (PFA) (USB Corporation) for 5-10 min, permeabilized in PBS with fish skin gelatin (Sigma-Aldrich) and Triton X-100 (ICN Biomedicals) (PBS/FSG/Triton) and blocked in 10% normal goat serum (Gibco). The fixed 2-D and 3-D cultured cells were equally stained with primary antibodies for 1 h, washed 3 times in PBS and then stained with corresponding secondary antibodies for 45 min. Nuclear stains were combined with the secondary antibodies at a concentration of 0.05 μg/ml. Primary antibodies used included anti-N-myc, HuD, Bcl-2, Bax and Bak (Santa Cruz Biotechnology). Alexa-488-conjugated secondary antibodies, and the To-Pro nuclear stains were from Invitrogen. Propidium Iodide (PI) (Sigma-Aldrich) was used as an alternative nuclear stain. Imaging was performed using a Leica TCS SP2 confocal microscope equipped with three lasers (Leica Microsystems). Six to eighteen 0.2-μm optical slices per image were collected at 512×512 pixel resolution. The pinhole size, gain and contrast, filter settings, and laser output were held constant for each comparison of the 2-D and 3-D image sets.

Western Blot Analysis

Cells were lysed on ice for 10 min using buffer (0.15 M NaCl, 5 mM EDTA, pH 8, 1% Triton X-100, 10 mM Tris-HCl, pH 7.40) supplimented with 5 mM dithiothreitol and a Protease Inhibitor Cocktail for mammalian cells (Sigma-Aldrich). Protein concentrations were measured with the BCA assay (Pierce Biotechnology). After optimization for each sample, total protein (40 μg/lane for N-myc, HuD, Bcl-2, and Bak, and 50 μg/lane for Bax) was resolved in 12% Tris-HCl pre-cast gels (BioRad), and electrophoretically transferred to nitrocellulose Protran membranes (Schleicher and Schuell BioSciences). Non-specific binding was blocked with 3% BSA fraction V (Sigma-Aldrich) in PBS-Tween (PBST) at 4° C. over night. Target proteins were detected with rabbit or mouse primary antibodies for 2 h at room temperature or at 4° C. over-night (all antibodies were from Santa Cruz Biotechnology except for β-actin (Abcam). The blots were washed 3 times in PBST and incubated for 45 min with horseradish peroxidase-conjugated anti-rabbit or anti-mouse secondary antibodies (Santa Cruz Biotech.) The blots were again washed 3 times in PBST, developed for 1-2 min in Western Blot Luminol Reagent (Santa Cruz Biotechnology) and visualized using a Kodak Imager 2000 and Kodak Image Analysis Software.

Apoptosis Assays

SY cells (1×10⁶) cultured in 2-D or 3-D were incubated with or without 10 nM TG. The 2-D and 3-D cells were harvested using trypsin, washed in PBS, and fixed for 5-10 min in 2% PFA. Prior to fixation, the 3-D-cultured cells treated inside of the RWV were separated from the beads using a 40-μm cell strainer (Becton Dickinson). The fixed cells were permeabilized in PBS/FSG/Triton and blocked with 10% NGS. Apoptosis was evaluated using the Apoptag TUNEL assay kit (Chemicon). The results were analyzed using a Leica TCS SP2 confocal microscope as described above. Cell morphology consistent with apoptosis including cell shrinkage, nuclear condensation and membrane blebbing were assessed along with the fluorescein staining for TUNEL. The number of apoptotic cells counted was divided by the total (500 minimum) number of cells counted. This protocol was also followed for evaluation of apoptosis in PC12 cells. An increased drug tolerance, 30-nM TG was used in the PC12 assay. 3-D-cultured PC12 samples were stimulated for 5 days after removal from the RWV to multi-well dishes.

Microarray Analysis

Microarray experiments and analysis of data was performed according to previously described protocols (Kaushal D, C. W. N. Analyzing and Visualizing Expression Data with Spotfire. Current Protocols in Bioinformatics 2004; Tekautz T M, Zhu K, Grenet J, Kaushal D, Kidd V J, Lahti J M. Evaluation of IFN-gamma effects on apoptosis and gene expression in neuroblastoma—preclinical studies. Biochimica et biophysica acta, 2006; 1763: 1000-10). Microarray experiments utilized the 44,544 70-mer element Human Exonic Evidence based Oligonucleotide (HEEBO) microarray, supplied by the Stanford Functional Genomics Facility. RNA was isolated from approximately 5×10⁶ 2-D and 3-D cultured cells using an RNeasy kit (Qiagen) plus DNA-free (Ambion), to eliminate DNA contamination. Five micrograms of mRNA was used to incorporate Cy3 (2-D samples) or Cy5 (3-D samples). Labeling, hybridization and scanning utilized previously described protocols (Tekautz et al., 2006). The resulting text data was imported into Spotfire DecisionSite (Spotfire Inc), filtered, and subjected to statistical analysis (Kaushal and Naeve, 2004). Genes whose expression changed by 1.5 fold (with a corrected t-test P<0.05) were considered to be differentially expressed in a statistically significant manner. Pathway analysis was performed by uploading significant dataset(s) into Ingenuity Pathways Analysis algorithm. Pathways that were perturbed in a statistically significant manner (P<0.05) were included in analysis.

Microarray data are annotated both in terms of universal gene symbols (Gene Symbol) and known gene function (Gene Description). Microarray experiments were performed on three biologically replicate Human Exonic Evidence-based Oligonucleotide arrays (#s 53383, 53384 and 52791). Differentially expressed genes were selected on the basis of statistical significance using one-way analysis of variance (P value) and magnitude of change in gene expression on a log₂ scale (M). A magnitude change of 50% (1.5-fold) along with P<0.05 was considered significant.

QRT-PCR

RNA was collected as for the microarray analysis. The QuantiFast SYBR Green RT-PCR kit (Qiagen) was used for the QRT-PCR. All assays were performed as per manufacturer's instruction with Qiagen QuantiTect primer pairs in a 96-well block ABI 7700 RT cycler.

Human SH-SY5Y neuroblastoma cells (American Type Culture Collection ATCC CRL-2266) were maintained in complete growth medium (1:1 mixture of Dulbecco's Modified Eagle Medium (D-MEM 11791 Gibco/Invitrogen, Carlsbad, Calif. “Gibco” hereafter) and Ham F-12 Medium (Ham F-12 11765, Gibco), 10% Fetal Bovine Serum (defined FBS Hyclone, Logan, Utah), 1.0 mM sodium pyruvate (supplied in the D-MEM), 0.1 mM non-essential amino acids (MEM NEAA 100×11140, Gibco), 1.5 g/L sodium bicarbonate (7.5% solution 25080, Gibco) within a 5%-CO₂ infused air atmosphere incubator (VWR 2400) at 37° C. The cells were originally seeded as standard monolayers (ML) into T75 culture flasks (Corning, Fisher Scientific, Pittsburgh, Pa.) with medium renewal every 3-7 days. Subculture and freezing of cells were performed following the procedures listed in the ATCC product sheet.

Growth medium was supplemented with 1× of the following antibiotic/antimycotic products: Penicillin/Streptomycin (100× 15140-122, Gibco) and Amphotericin (100×15240-062, Gibco). Trypsin/EDTA (2.5% 25200056, Gibco) was used to dislodge the cells for subculture. DMSO (D2650, Sigma) 5% v/v was added to the cryoprotectant medium used for storage of frozen cell stocks. Trypan Blue (15250-061, Gibco), in a 1:1 ratio with trypsinized and resuspended cells was employed in counting, subculture and viability assays.

Cytodex-3 Collagen-Coated Microcarrier Beads (Amersham Biosciences 17-0485-01) were reconstituted to 1.0 g/50 ml in sterile phosphate buffered saline solution (PBS) as per the manufacturer's instructions. Before being added to cell culture the beads were “pre-conditioned,” as follows: 10 ml of the mixture was extracted into a sterile 50-ml conical tube and allowed to settle. Excess PBS was removed and the remaining bead slurry was pre-warmed to 37° C. The beads were then packaged at approximately 3×10⁶ beads/gram dry weight. High Aspect Ratio Vessels (HARV D-405 disposable vessels), single rotator bases and power supply units were purchased from Synthecon, Inc., Houston, Tex. Five and 10-cc luer-lock disposable sterile syringes (Exel 14-841-54 and Exel 14-841-54, Fisher Scientific, Pittsburgh, Pa.) were used for culture sampling, drug or reagent administration and to dislodge any bubbles in the system.

Fifty-milliliter disposable HARV vessels were filled to approximately 70% with pre-warmed complete medium. One 5-cc and one 10-cc sterile syringe were attached to the side ports of the HARV and filled with 2-5 ml of complete medium. Medium addition and renewal were performed through the main port.

SH-SY5Y cells cultured in 2-D were allowed to reach approximately 80% confluency in T75 culture flasks. At this point the growth medium was removed. The cells were dislodged with trypsin/EDTA, resuspended in complete growth medium and removed from the flask. Trypan Blue was used to monitor viability of the cells during counting in a hemocytometer (Bright-Line Reichert Scientific, Buffalo, N.Y.). Approximately 10⁷ viable SH-SY5Y cells were combined with an aliquot of pre-conditioned Cytodex-3 beads, and loaded into the HARV through the main port. Additional pre-warmed medium was added to completely fill-up the vessel. The HARV was attached to a rotator base and power supply. Initial speed was set at 18-20 rpm based on observed sedimentation. Continuous formation of aggregates in the HARV would then determine subsequent rpm settings (typically 18-22 rpm). Sedimentation rates and bubble formation were monitored and addressed daily.

Droplet samples of the culture were removed every few days to observe changes in cell morphology, adherence to the beads, viability, etc. The bulk of the 3-D culture was allowed to remain in the HARV for 3-4 weeks, when larger aliquots of the cells would be removed for experimental procedures.

In the resulting 3D versus monolayer (ML) culture, neuronal SH-SY5Y cells underwent distinct morphological changes as revealed by scanning electron and confocal microscopy, and also revealed unexpected phenotypic changes. Expression of the proto-oncogene N-myc and its RNA building protein HuD was decreased in 3D culture as compared to standard ML conditions. The neuronal cell culture showed a decline in the anti-apoptotic protein Bcl-2 in 3D culture, coupled with increased expression of the pro-apoptotic proteins BAX and BAK. Using microarray analysis, significantly differing mRNA levels for an additional 40 genes in the cells of each culture type were found. Moreover, thapsigarin-induced apoptosis was notably enhanced in the 3D cultured SH-SY5Y cells. Comprehensively, these results indicate that a 3D culture approach may begin to close the phenotypic gap between transformed neuronal cell lines and untransformed neurons and that it may readily be used for in vitro research of neuronal pathogenesis in the central nervous system.

### EXAMPLE 1

### 3-D Culture Changes the Morphology and Proliferation Rate in SY Neuronal Cells

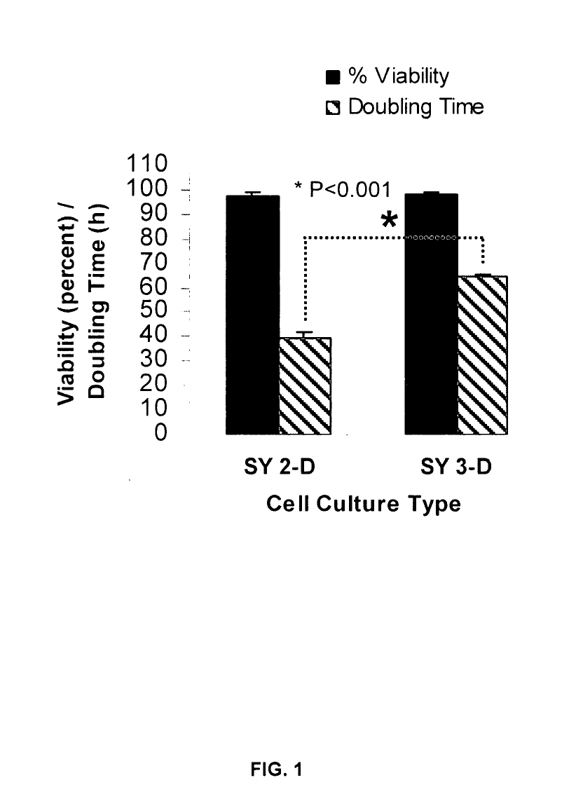

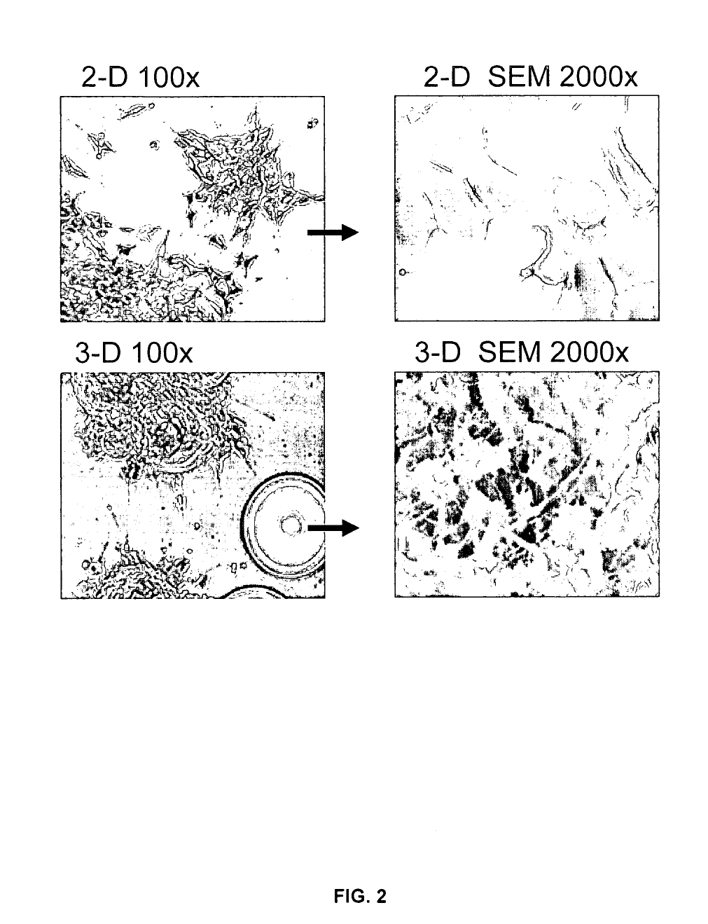

SY cells cultured for 21 days in the RWV, and then for counting purposes transferred back to 2-D culture flasks for 5 days, revealed a decrease in the cell doubling rate from 40 h to approximately 65 h, with no change in cell viability (FIG. 1). Thus, the 3-D phenotype of SY cells comprises a decrease in the cell doubling rate. Because the carrier beads used in the 3-D culture were coated in collagen, additional SY cells were cultured for 3 weeks and for 4 weeks in 2-D flasks coated with collagen. No detectable difference was observed in the morphology, cell viability or doubling rate of 2-D cells cultured on plastic as compared to collagen. Scanning electron microscopy (SEM) revealed important differences in the morphology of SY cells cultured in 2-D or in 3-D. Specifically, only the 3-D-cultured SY cells acquired a parental, tissue-like conformation with dramatic increases in neurite extension, direction and number (FIG. 2). Thus, the 3-D phenotype of SY cells further comprises parental, tissue-like conformation with dramatic increases in neurite extension (outgrowth), direction and number.

### EXAMPLE 2

### Decreased Expression of N-myc and HuD

Human neuroblastoma cells are typically characterized by de-differentiation. They have re-entered S-phase of the cell cycle, and are highly resistant to apoptosis (Kang et al., 2006; van Noesel et al., 2003). Amplified expression of the proto-oncogene N-myc has been correlated with cellular de-differentiation and increased resistance to apoptosis, and is believed to have a crucial role in maintenance of the cells' malignant phenotype (Chagnovich and Cohn, 1996; Grandinetti et al., 2006; Smith et al., 2004; van Golen et al., 2003). The RNA binding protein HuD functions in stabilizing N-myc mRNA and may consequently enhance steady-state expression levels of this oncogene (Chagnovich and Cohn, 1996; Grandinetti et al., 2006; Lazarova et al., 1999). Reduced expression of the HuD protein could therefore contribute, through destabilization of N-myc, to an increase in cellular differentiation.

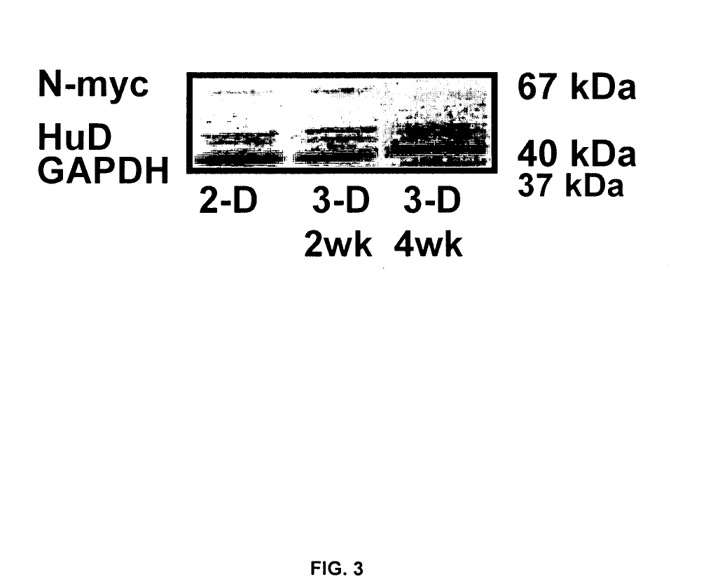

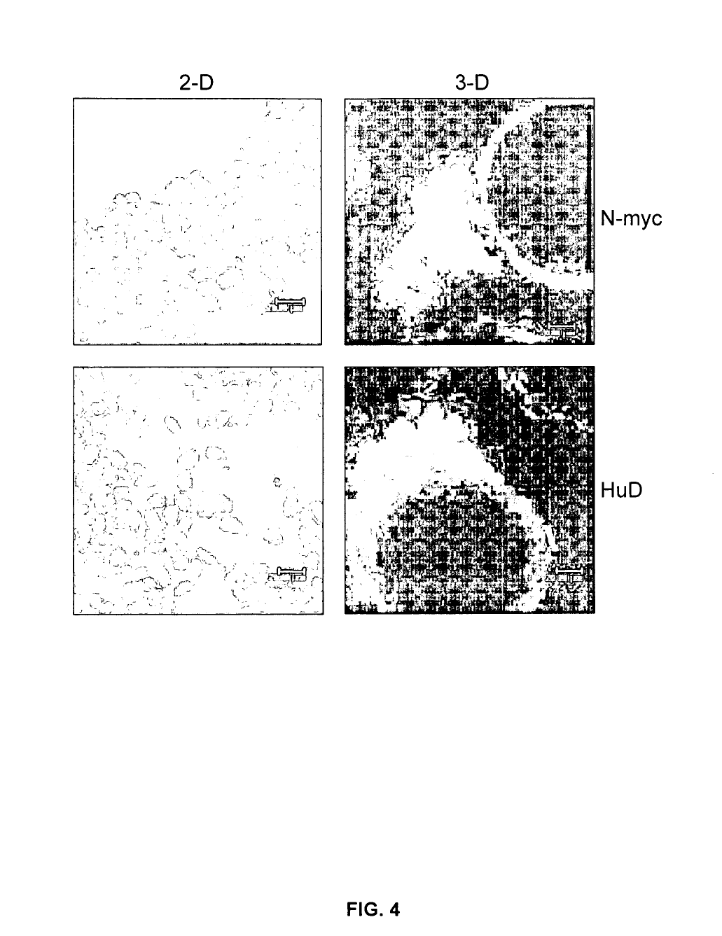

Western analysis confirmed a culture-dependent shift in protein expression of these markers, with the decrease positively aligning with the length of time the cells had spent in 3-D culture (FIG. 3). Images obtained with confocal microscopy revealed a diminished level of N-myc and HuD protein expression in SY cells cultured in 3-D as opposed to 2-D (FIG. 4). Thus, the 3-D phenotype of SY cells further comprises reduced expression of N-myc and HuD proteins.

### EXAMPLE 3

### Apoptosis Resistance is Diminished in 3-D Cultured SY and PC12 Cells

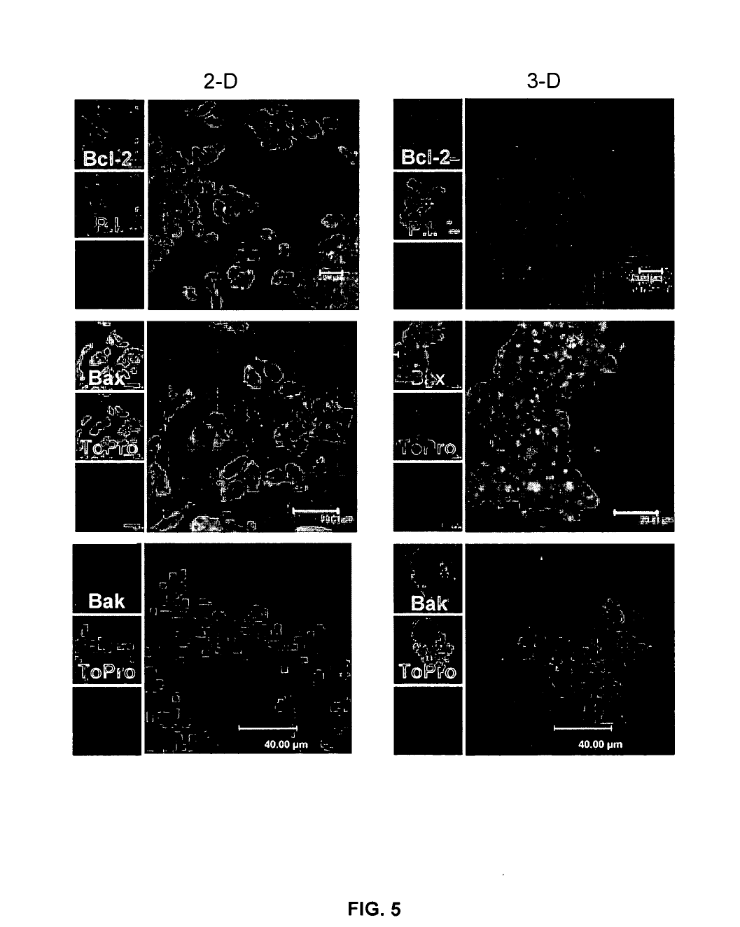

Cells over-expressing the anti-apoptotic protein Bcl-2 or cells with depleted pro-apoptotic Bax and Bak exhibit resistance to cell death as induced by mitochondrial dysfunction and ER stress (Elyaman W, Terro F, Suen K C, Yardin C, Chang R C, Hugon J. BAD and Bcl-2 regulation are early events linking neuronal endoplasmic reticulum stress to mitochondria-mediated apoptosis. Brain research, 2002; 109: 233-8; Henshall D C, Araki T, Schindler C K, Lan J Q, Tiekoter K I, Taki W, Simon R P. Activation of Bcl-2-associated death protein and counter-response of Akt within cell populations during seizure-induced neuronal death. J Neurosci, 2002; 22: 8458-65; Murakami Y, Aizu-Yokota E, Sonoda Y, Ohta S, Kasahara T. Suppression of endoplasmic reticulum stress-induced caspase activation and cell death by the overexpression of Bcl-xL or Bcl-2. Journal of biochemistry, 2007; 141: 401-10; Scorrano L, Oakes S A, Opferman J T, Cheng E H, Sorcinelli M D, Pozzan T, Korsmeyer S J. BAX and BAK regulation of endoplasmic reticulum Ca2+: a control point for apoptosis. Science (New York, N.Y., 2003; 300: 135-9). Because increased resistance to apoptosis is one hallmark of a transformed phenotype in many cancer cell lines, it was important to assess the effects of 3-D culture on the expression of key proteins in the apoptosis pathway. The present invention discloses a decreased expression of Bcl-2 coupled with increased Bax and Bak proteins in 3-D cultured SY cells as compared to those cultured in standard 2-D conditions (FIGS. 5 & 6). While confocal imaging clearly indicated increased Bak protein in 3-D cultured cells, Western analysis was not sensitive enough to detect its expression.

The next consideration was to assess apoptosis functionally and to confirm that the findings were not restricted to a single cell line. PC12 is a rat pheochromocytoma cell line that can be stimulated with nerve growth factor to differentiate into sympathetic-like neurons (Greene L A, Tischler A S. Establishment of a noradrenergic clonal line of rat adrenal pheochromocytoma cells which respond to nerve growth factor. Proceedings of the National Academy of Sciences of the United States of America, 1976; 73: 2424-8). Due to their induced ability to cease division, become electrically excitable and extend neurites, PC12 cells have become an extremely well characterized in vitro model for studies of neuronal differentiation and survival (Attiah D G, Kopher R A, Desai T A. Characterization of PC12 cell proliferation and differentiation-stimulated by ECM adhesion proteins and neurotrophic factors. Journal of materials science, 2003; 14: 1005-9; Das P C, McElroy W K, Cooper R L. Differential modulation of catecholamines by chlorotriazine herbicides in pheochromocytoma (PC12) cells in vitro. Toxicol Sci, 2000; 56: 324-31; Lelkes et al., 1998; Ulloth J E, Almaguel F G, Padilla A, Bu L, Liu J W, De Leon M. Characterization of methyl-beta-cyclodextrin toxicity in NGF-differentiated PC12 cell death. Neurotoxicology, 2007; 28: 613-21; Vyas S, Juin P, Hancock D, Suzuki Y, Takahashi R, Triller A, Evan G. Differentiation-dependent sensitivity to apoptogenic factors in PC12 cells. The Journal of biological chemistry, 2004; 279: 30983-93).

Thapsigargin (TG) is known to induce apoptosis through the passive release of Ca²⁺ from ER stores. These events lead to subsequent increases in cytosolic Ca²⁺, stressing both the ER and the mitochondria (Elyaman et al., 2002; Nechushtan A, Smith C L, Lamensdorf I, Yoon S H, Youle R J. Bax and Bak coalesce into novel mitochondria-associated clusters during apoptosis. The Journal of cell biology, 2001; 153: 1265-76; Nguyen H N, Wang C, Perry D C. Depletion of intracellular calcium stores is toxic to SH-SY5Y neuronal cells. Brain Res, 2002; 924: 159-66; Scorrano et al., 2003; Zong W X, Li C, Hatzivassiliou G, Lindsten T, Yu Q C, Yuan J, Thompson C B. Bax and Bak can localize to the endoplasmic reticulum to initiate apoptosis. The Journal of cell biology, 2003; 162: 59-69). In order to determine inherent differences in apoptosis between the 3-D and 2-D cultured cells, the terminal uridine deoxynucleotidyl transferase dUTP nick end labeling (TUNEL) assay was used. SY cells were incubated with 10-nM TG for 24 hours and for 5 days. The 3-D-cultured SY cells were treated either inside the RWV (3-D(RWV) or after transfer back into standard culture flasks (3-D). Additionally, PC12 cells were incubated with 30-nM TG, for 5 days. All of the 3-D-cultured PC12 cells were treated after transfer back into standard culture flasks. The SY and PC12 cells grown in 2-D culture were treated in their respective dishes.

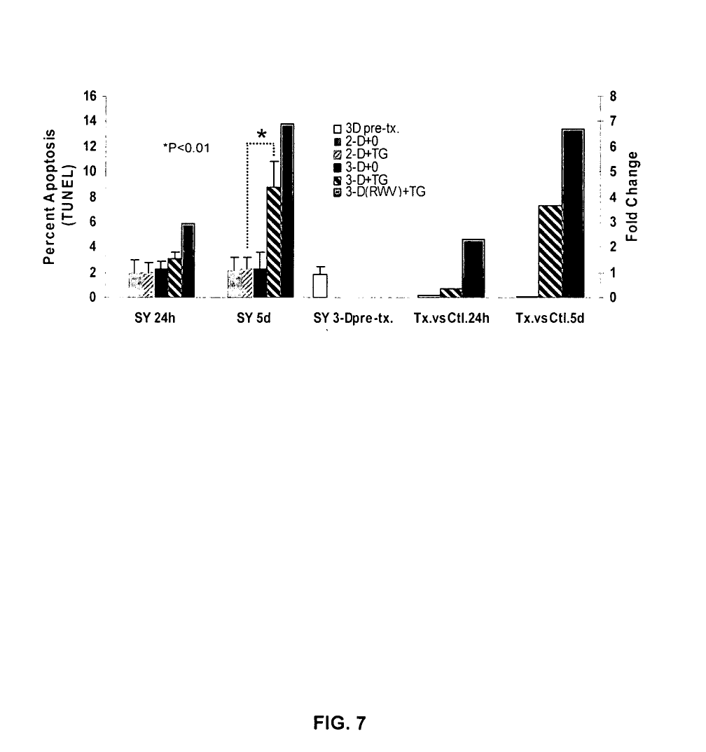

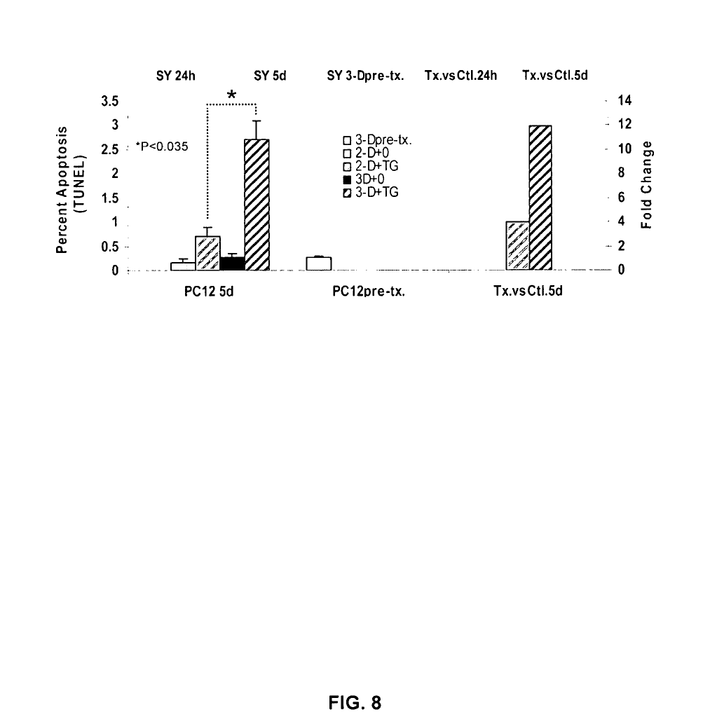

In a 5-day comparison of TG-stimulated versus non-stimulated control cells, an approximate 4- to 7-fold increase in the occurrence of apoptosis was observed in 3-D as opposed to 2-D culture (FIG. 7). In a similar 5-day comparison, 3-D cultured PC12 cells were approximately 3-fold more susceptible to apoptosis than were the 2-D cells (FIG. 8). At 24 h, a noticeable difference in the degree of apoptosis occurring in stimulated versus control cells was found only in the 3-D(RWV) cells (FIGS. 7 & 8).

Thus, the 3-D phenotype of SY cells further comprises decreased expression of Bcl-2 protein, increased expression of Bax and Bak proteins, and the 3-D phenotypes of both SY cells and PC12 cells comprise increased susceptibility to pro-apoptotic signals (increased sensitivity to apoptosis).

### EXAMPLE 4

SY cells maintain 3-D culture-induced alterations in the phenotypic markers N-myc and Bcl-2 for at least 5 days after return to 2-D culture

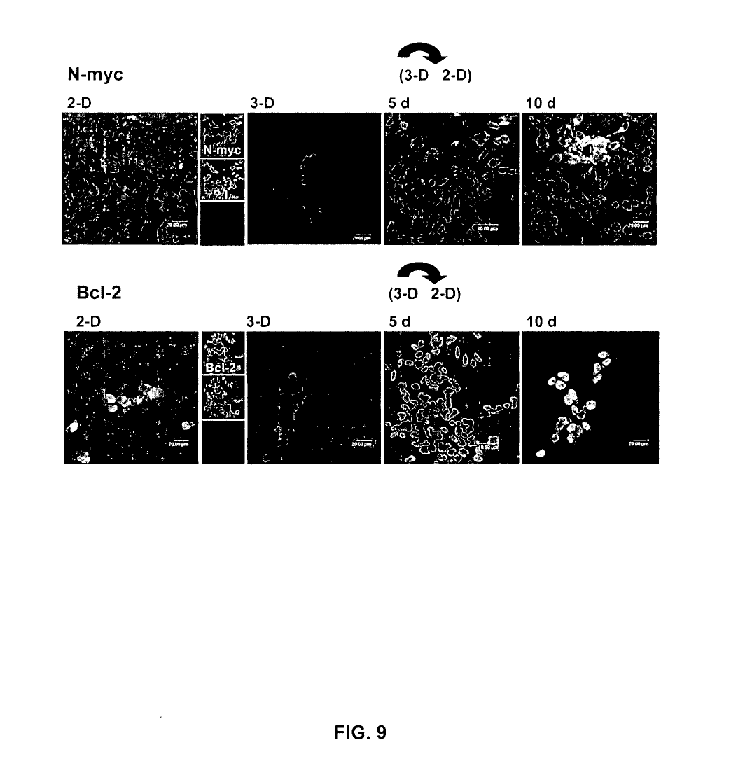

As many studies of neuronal pathogenesis involve co-cultures of neuronal cell lines with primary glia and/or other live organisms propagated in 2-D culture, it was important to evaluate the length of time that SY cells from 3-D culture would retain a 3-D phenotype once they were transferred back into 2-D culture. Thus, the expression of N-myc and Bcl-2, two molecular markers closely related to both differentiation and tumorigenicity, were examined (Elyaman et al., 2002; Fan et al., 2001; Kang et al., 2006; Pregi N, Vittori D, Perez G, Leiros C P, Nesse A. Effect of erythropoietin on staurosporine-induced apoptosis and differentiation of SH-SY5Y neuroblastoma cells. Biochimica et biophysica acta, 2006; 1763: 238-46; Ribas and Boix, 2004; Smith et al., 2004; van Golen et al., 2003; van Noesel et al., 2003). Assessment of the SY cells that had been “pre-conditioned” in 3-D culture for approximately 3 wk and were then removed to 2-D culture revealed a 5-day experimental window during which both N-myc and Bcl-2 protein expression remained suppressed, indicating that reversion of the 3-D culture-induced changes was minimal (FIG. 9). Thus, the 3-D phenotype of SY cells further comprises retention of the 3-D phenotype for up to 5 days following removal from 3-D culture and subsequent transfer to 2-D culture.

### EXAMPLE 5

### Microarray Analysis of Gene Expression in SY Cells Cultured in 3-D and in 2-D

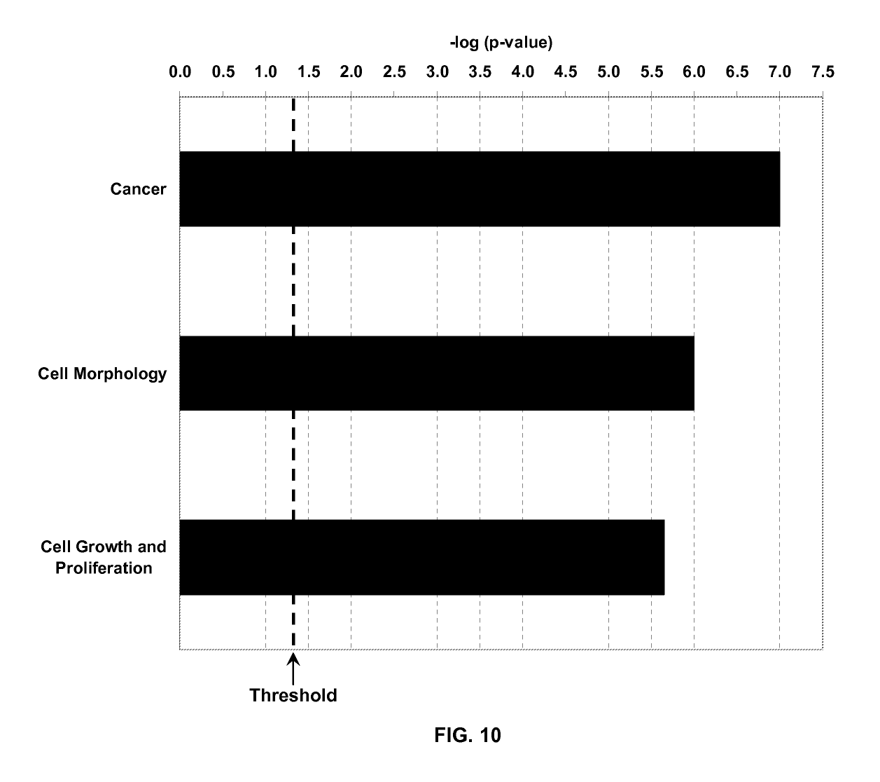

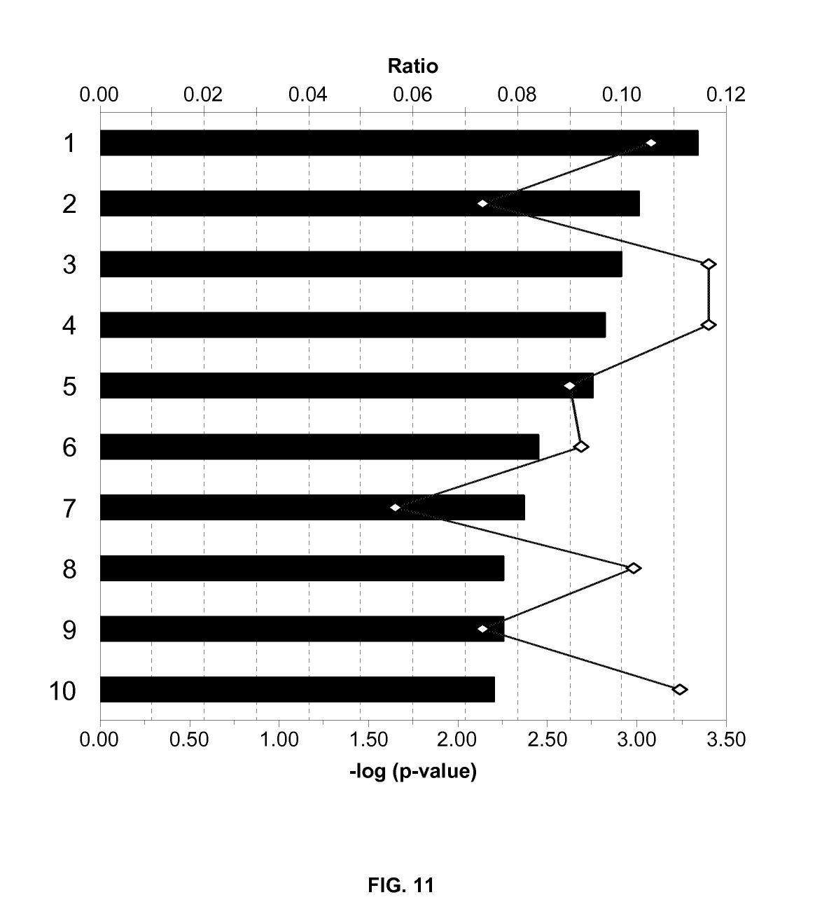

In an effort to expand and further clarify the above findings related to the differing states of differentiation and morphology between 2-D and 3-D-cultivated SY cells (i.e., to further characterize the phenotype of 3-D-cultivated cells), microarray analysis was employed to observe the culture-induced effects on global gene expression. Because abnormalities in the expression and activity of multiple genes often work in concert to effect a transformed cellular phenotype (Hanahan D, Weinberg R A. The hallmarks of cancer. Cell, 2000; 100: 57-70; Li et al., 2006; Park and Lee, 2003; Tweddle D A, Malcolm A J, Cole M, Pearson A D, Lunec J. p53 cellular localization and function in neuroblastoma: evidence for defective G(1) arrest despite WAF1 induction in MYCN-amplified cells. The American journal of pathology, 2001; 158: 2067-77), Ingenuity Pathways Analysis (IPA) software was used to compare the mRNA levels in 44,544 70-mer oligos corresponding to over 24,000 human genes. Cancer, cell morphology and proliferation pathways were among those found to be the most altered (FIG. 10). The G1/S and G2/M cell cycle check points, as well as the p53 and neuregulin signaling pathways, were also significantly affected (FIG. 11).

Along with abnormalities in the p53 tumor suppressor gene pathway, dysregulation of the cell cycle is one of the most frequent alterations found in tumor development, with the inappropriate progression of G1/S being especially common (Kuipper R P, Schoenmakers E F, van Reijmersdal S V, Hehir-Kwa J Y, van Kessel A G, van Leeuwen F N, Hoogerbrugge P M. High-resolution genomic profiling of childhood ALL reveals novel recurrent genetic lesions affecting pathways involved in lymphocyte differentiation and cell cycle progression. Leukemia, 2007; 21: 1258-66; Park and Lee, 2003; Tweddle et al., 2001; Zhu et al., 1999). In the normal dividing cell, cyclin-dependent kinases (CDKs) form a complex with D/E-type cyclins to phosphorylate the retinoblastoma (Rb) gene, causing the release of bound E2F-family transcription factors. These now unbound E2F proteins then act to drive G1/S phase transition by the activation (or repression) of multiple gene targets affecting cellular growth and proliferation, nucleotide metabolism and DNA synthesis (Ebelt H, Hufnagel N, Neuhaus P, Neuhaus H, Gajawada P, Simm A, Muller-Werdan U, Werdan K, Braun T. Divergent siblings: E2F2 and E2F4 but not E2F1 and E2F3 induce DNA synthesis in cardiomyocytes without activation of apoptosis. Circulation research, 2005; 96: 509-17; Jiang Y, Saavedra H I, Holloway M P, Leone G, Altura R A. Aberrant regulation of survivin by the RB/E2F family of proteins. The Journal of biological chemistry, 2004; 279: 40511-20; L et al., 2006; Parisi T, Yuan T L, Faust A M, Caron A M, Bronson R, Lees J A. Selective requirements for E2f3 in the development and tumorigenicity of Rb-deficient chimeric tissues. Molecular and cellular biology, 2007; 27: 2283-93; Park and Lee, 2003). Histone deacetylases (HDACs) form a complex with bound E2F proteins and are also released upon phosphorylation of Rb. Importantly, HDAC inhibitors have been shown to cause cell cycle arrest in G1 and to function in cellular differentiation and apoptosis (Xiong Y, Zhang H, Beach D. Subunit rearrangement of the cyclin-dependent kinases is associated with cellular transformation. Genes & development, 1993; 7: 1572-83; Zhou Q, Melkoumian Z K, Lucktong A, Moniwa M, Davie J R, Strobl J S. Rapid induction of histone hyperacetylation and cellular differentiation in human breast tumor cell lines following degradation of histone deacetylase-1. The Journal of biological chemistry, 2000; 275: 5256-63). Because of its strong ties to transformation, the actual variance reported in the G1/S pathway was examined closely.

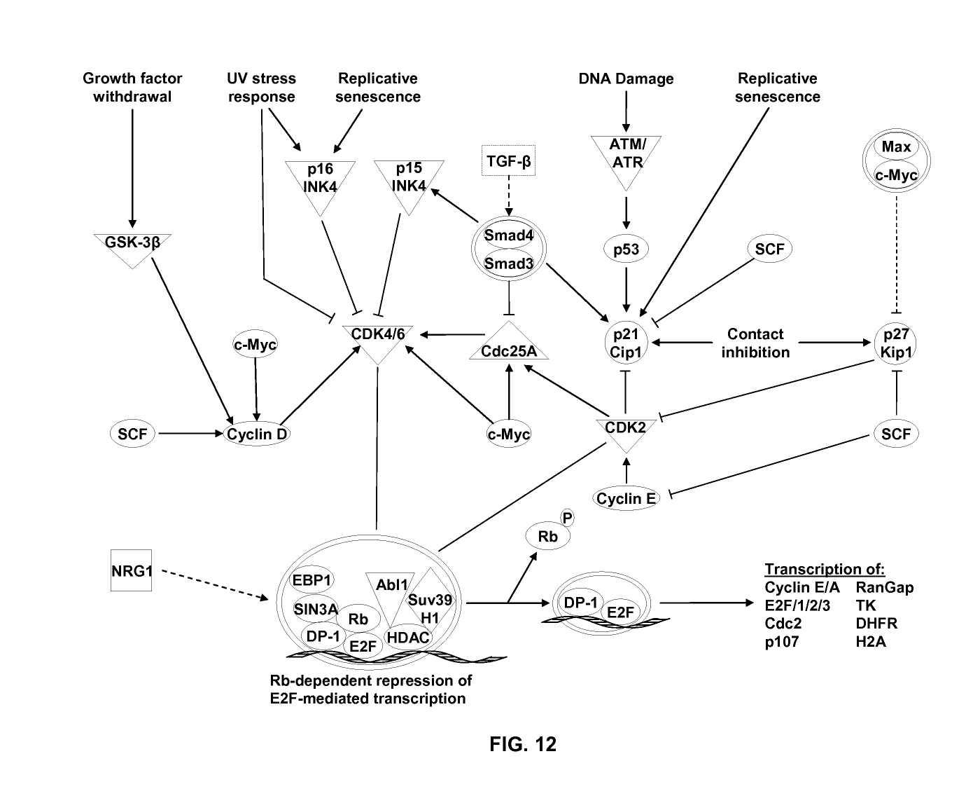

The CDK4/6 inhibitor CDKN2B was found to be significantly up-regulated in 3-D versus 2-D cultured SY cells. At the same time, the transcription factor E2F3, HDAC2 and the neuregulin1 (NRG1) gene, whose product promotes growth and proliferation in neuronal cells of the peripheral and central nervous systems (Fallon K B, Havlioglu N, Hamilton L H, Cheng T P, Carroll S L. Constitutive activation of the neuregulin-1/erbB signaling pathway promotes the proliferation of a human peripheral neuroepithelioma cell line. Journal of neuro-oncology, 2004; 66: 273-84; Rieff H I, Raetzman L T, Sapp D W, Yeh H H, Siegel R E, Corfas G. Neuregulin induces GABA(A) receptor subunit expression and neurite outgrowth in cerebellar granule cells. J Neurosci, 1999; 19: 10757-66), were each significantly down-regulated (FIG. 12). These events clearly indicate arrest in G1. Rb gene expression was also decreased, but without knowing the phosphorylation state of this gene, correlation with the cell cycle is questionable.

### EXAMPLE 6

### RT-PCR Confirms the Differential Expression of G1/S Cell-Cycle Check Point Genes in 3-D Versus 2-D Cultured SY Cells

A significant part of the microarray analysis was focused on exploring culture-induced differential gene expression in a neuronal cell line that could indicate phenotypic reversion toward a more normal state. Pathways such as growth and proliferation or the cell cycle checkpoints were of interest. RT-PCR was used to confirm the initial array findings. In order to maintain integrity in this experiment as compared to the microarray analysis, aliquots of the same SY 3-D and 2-D cell RNA that was collected for each of the arrays were used. Expression changes in 3 of the 4 selected genes known to influence the G1/S cell cycle checkpoint matched the microarray data, as shown in TABLE 1. Values were obtained using IPA software, version 5.0. Minimum fold change≧1.5.

The array results were confirmed with QRT-PCR, as shown in TABLE 2 (“*” indicates P<0.05). Reactions were run in triplicate with GADPH gene expression used as the reference. PCR inefficiencies, average fold change, and statistical analyses were performed using the REST© software program. All genes in this pathway were represented on the chips. For both the microarray analysis of TABLE 1 and the QRT-PCR confirmation of TABLE 2, mRNA was collected at passage 8 (2-D and 3-D cultures) with n=2 for each culture type.

TABLE 1
Microarray analysis results for genes involved in G1/S cell cycle progression

| HUGO | | | | | Entrez |
|---|---|---|---|---|---|
| Gene | | Log | | | Gene ID |
| Symbol | Description | Ratio | Location | Type | (H) |
| CDKN2B | cyclin-dependent kinase | +3.348 | nucleus | transcription | 1030 |
| | inhibitor 2B (INK4, p15, | | | regulator | |
| | inhibits CDK4) | | | | |
| E2F3 | E2F transcription factor 3 | −2.15 | nucleus | transcription | 1871 |
| | | | | regulator | |
| HDAC2 | histone deacetylase 2 | −2.236 | nucleus | transcription | 3066 |
| | | | | regulator | |
| NRG1 | neuregulin 1 | −4.403 | nucleus | extracellular | 3084 |
| | | | | space | |
| RB1 | retinoblastoma 1 (including | −1.574 | nucleus | transcription | 5925 |
| | osteocarcinoma) | | | regulator | |
| SKP1A | S-phase kinase-associated | −1.325 | nucleus | transcription | 6500 |
| | protein 1A (p19A) | | | regulator | |

TABLE 2
QRT-PCR confirmation of TABLE 1 microarray results

| | 3-D | |
|---|---|---|
| Gene | (fold change) | P-value |
| *CDKN2B | +4.04 | 0.001 |
| E2F3 | +1.00 | 0.947 |
| *HDAC2 | −1.57 | 0.050 |
| *NRG1 | −2.39 | 0.001 |

Since similar results were observed with both SY cells and PC12 cells, a person of ordinary skill in the art may reasonably assume that the results described herein are applicable to most if not all transformed neuronal cell lines (i.e., any transformed neuronal cell line cultured via the 3-D culture methods disclosed herein would likely exhibit an analogous 3-D phenotype).

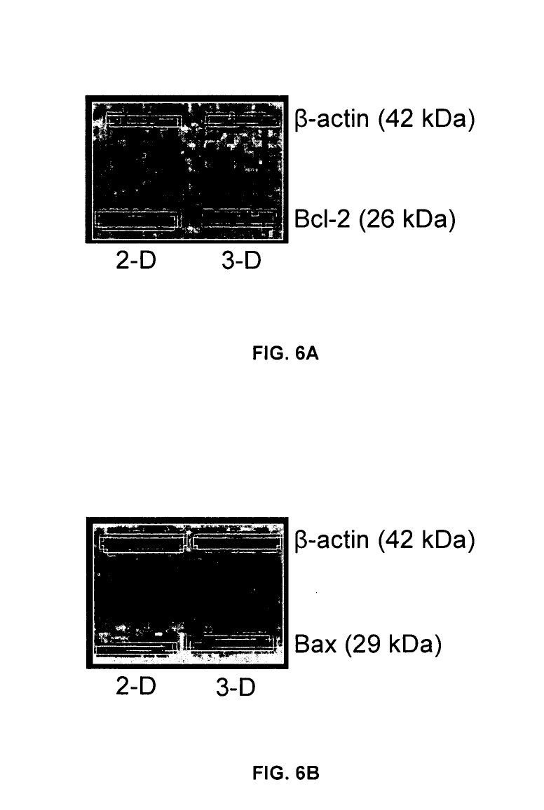

The present invention discloses culture-induced changes in the morphology and biomarker expression of 3-D-cultured SY cells, reflecting a more differentiated, and thus a less transformed, phenotype. The invention also discloses that apoptosis resistance of 3-D-cultured SY and PC12 cells is diminished (FIGS. 3-8), and that the doubling rate of SY cells cultured in 3-D declines while retaining viability (FIG. 1). Microarray analysis comparing 3-D and 2-D-cultured SY cells indicates strongly that alterations in G1/S cell cycle progression mechanisms contribute to the diminished doubling rate observed in 3-D-cultured SY cells (TABLE 1). Neuronal cells arrested at this checkpoint are known to either return to G0 and re-differentiate, or die by apoptosis (Becker and Bonni, 2004). Due to the decline in doubling rate and the near-100 percent viability of the 3-D-cultured SY cells, it is reasonable to assume that the cells were returning to quiescence. Confirmation of the array results involved in this pathway was obtained using quantitative real-time (QRT)-PCR (TABLE 2). Lending added support to the observation that 3-D-cultured SY cells represent a more differentiated—and thus a less transformed—phenotype, culture-induced variance in several other prominent pathways known to be correlated with transformation and cancer were also identified on the microarray (TABLE 3). The microarray data of TABLE 3 are annotated both in terms of universal gene symbols (Gene Symbol) and known gene function (Gene Description). Microarray experiments were performed on three biologically replicate Human Exonic Evidence-Based Oligonucleotide (HEEBO) arrays (#s 53383, 53384 and 52791). Differentially expressed genes were selected on the basis of statistical significance using one-way analysis of variance (P value) and magnitude of change in gene expression on a log₂ scale (M). A magnitude change of 50% (1.5-fold) along with P<0.05 was deemed significant.

TABLE 3

| Gene | | | 53383 | 53384 | 52791 | |
|---|---|---|---|---|---|---|
| Symbol | Gene Description | p value | (M) | (M) | (M) | Average M |
| GNAS | GNAS complex locus | 5.94E−03 | 4.9146 | 4.2084 | 6.7293 | 5.2841 |
| FOS | V-fos FBJ murine osteosarcoma | 1.96E−06 | 4.0031 | 3.9919 | 7.7385 | 5.2445 |
| | viral oncogene homolog | | | | | |
| FOSB | FBJ murine osteosarcoma viral | 5.18E−03 | 2.9227 | 3.3780 | 8.0979 | 4.7995 |
| | oncogene homolog B | | | | | |
| GEM | GTP binding protein | 1.56E−03 | 4.1920 | 3.8729 | 6.0612 | 4.7087 |
| | overexpressed in skeletal muscle | | | | | |
| LOC286411 | Hypothetical protein | 2.17E−03 | 4.0405 | 3.6798 | 6.1844 | 4.6349 |
| | LOC286411 | | | | | |
| EGR4 | Early growth response 4 | 6.32E−07 | 4.1942 | 4.2008 | 4.1887 | 4.1946 |
| SNCG | Synuclein | 4.22E−03 | 3.1706 | 2.7826 | 5.8755 | 3.9429 |
| LOC399851 | Hypothetical gene supported by | 7.54E−03 | 4.9741 | 4.0287 | 2.7278 | 3.9102 |
| | AY129010 | | | | | |
| RBBP8 | Retinoblastoma binding protein 8 | 1.65E−04 | 3.7846 | 3.6887 | 3.7405 | 3.7379 |
| C16orf35 | Chromosome 16 open reading | 1.53E−03 | 4.3341 | 4.0077 | 2.8568 | 3.7329 |
| | frame 35 | | | | | |
| FES | Feline sarcoma oncogene | 1.43E−03 | 4.0647 | 3.7681 | 3.2814 | 3.7047 |
| CYP4A11 | Cytochrome P450 | 1.05E−03 | 2.5335 | 2.7037 | 5.7357 | 3.6576 |
| STMN4 | Stathmin-like 4 | 1.42E−03 | 4.2550 | 3.9458 | 2.6045 | 3.6018 |
| CLCN3 | Chloride channel 3 | 5.56E−03 | 4.4882 | 3.8628 | 2.3713 | 3.5741 |
| NEUROG2 | Neurogenin 2 | 9.98E−03 | 0.7650 | 0.9362 | 8.9825 | 3.5612 |
| CBX3 | Chromobox homolog 3 (HP1 | 2.06E−03 | 4.7856 | 4.3699 | 1.2580 | 3.4712 |
| | gamma homolog | | | | | |
| LOC284454 | Hypothetical protein | 2.20E−02 | 1.3339 | 1.8076 | 7.1729 | 3.4381 |
| | LOC284454 | | | | | |
| ASS | Argininosuccinate synthetase | 5.16E−03 | 3.8439 | 4.4414 | 1.9665 | 3.4173 |
| HMGCR | 3-hydroxy-3-methylglutaryl- | 4.18E−04 | 4.1300 | 3.9645 | 2.1349 | 3.4098 |
| | Coenzyme A reductase | | | | | |
| SIRT5 | Sirtuin (silent mating type | 1.80E−03 | 4.7252 | 4.3400 | 1.0683 | 3.3778 |
| | information regulation 2 | | | | | |
| | homolog) 5 (S. cerevisiae) | | | | | |
| IRF2 | Interferon regulatory factor 2 | 1.72E−02 | 4.7961 | 3.6709 | 1.6388 | 3.3686 |
| UCN3 | Urocortin 3 (stresscopin) | 8.85E−03 | 1.6089 | 1.9455 | 6.4676 | 3.3407 |
| ZNF526 | Zinc finger protein 526 | 4.12E−02 | 2.3658 | 1.5464 | 6.0973 | 3.3365 |
| ANK3 | Ankyrin 3 | 2.91E−03 | 3.9058 | 3.5048 | 2.5943 | 3.3350 |
| C20orf91 | Chromosome 20 open reading | 2.01E−03 | 4.2833 | 3.9147 | 1.7784 | 3.3255 |
| | frame 91 | | | | | |
| NR4A1 | Nuclear receptor subfamily 4 | 8.26E−03 | 1.5105 | 1.2574 | 7.2071 | 3.3250 |
| TMC4 | Transmembrane channel-like 4 | 1.01E−05 | 2.3355 | 2.3206 | 5.2626 | 3.3062 |
| PGC | Progastricsin (pepsinogen C) | 9.16E−04 | 4.6601 | 4.3861 | 0.7513 | 3.2658 |
| RASSF4 | Ras association (RalGDS/AF-6) | 4.12E−05 | 4.1877 | 4.2418 | 1.2627 | 3.2307 |
| | domain family 4 | | | | | |
| ZCWPW2 | Zinc finger | 2.45E−02 | 3.2916 | 4.3230 | 2.0765 | 3.2304 |
| C1orf113 | Chromosome 1 open reading | 3.84E−03 | 4.6874 | 4.1386 | 0.8568 | 3.2276 |
| | frame 113 | | | | | |
| APOA5 | Apolipoprotein A-V | 4.36E−03 | 4.3167 | 3.6683 | 1.6606 | 3.2152 |
| GPR98 | G protein-coupled receptor 98 | 1.09E−03 | 4.0405 | 3.7817 | 1.7784 | 3.2002 |
| CRIP2 | Cysteine-rich protein 2 | 2.20E−03 | 4.6050 | 4.1913 | 0.7575 | 3.1846 |
| SNAPC2 | Small nuclear RNA activating | 2.42E−02 | 2.8780 | 3.9621 | 2.7090 | 3.1831 |
| | complex | | | | | |
| BLNK | B-cell linker | 1.90E−05 | 4.1300 | 4.1662 | 1.1752 | 3.1571 |
| LRRC37B | Leucine rich repeat containing | 3.43E−03 | 4.1391 | 3.6798 | 1.6494 | 3.1561 |
| | 37B | | | | | |
| UBXD5 | UBX domain containing 5 | 2.74E−03 | 3.7937 | 3.4157 | 2.1632 | 3.1242 |
| TTN | Titin | 3.89E−02 | 2.7651 | 3.6798 | 2.9236 | 3.1229 |
| LYPD3 | LY6/PLAUR domain containing 3 | 1.08E−03 | 4.1595 | 4.3298 | 0.8699 | 3.1197 |
| RPL10L | Ribosomal protein L10-like | 3.88E−04 | 4.0207 | 3.8653 | 1.3921 | 3.0927 |
| | Carboxylesterase 1 | | | | | |
| CES1 | (monocyte/macrophage serine | 8.35E−03 | 4.0623 | 3.3780 | 1.7944 | 3.0782 |
| | esterase 1) | | | | | |
| LOC391169 | Hypothetical LOC391169 | 3.61E−03 | 4.1942 | 3.7176 | 1.3157 | 3.0758 |
| HGF | Hepatocyte growth factor | 1.72E−02 | 1.0977 | 0.8399 | 7.2833 | 3.0737 |
| | (hepapoietin A; scatter factor) | | | | | |
| ACCN3 | Amiloride-sensitive cation | 1.82E−03 | 4.3360 | 3.9806 | 0.8949 | 3.0705 |
| | channel 3 | | | | | |
| LOC374395 | Similar to RIKEN cDNA | 4.02E−02 | 3.6469 | 2.1584 | 3.3907 | 3.0654 |
| | 1810059G22 | | | | | |
| RIPK4 | Receptor-interacting serine- | 1.79E−02 | 2.1462 | 2.0920 | 4.9505 | 3.0629 |
| | threonine kinase 4 | | | | | |
| LOC653073 | Similar to golgi autoantigen | 5.00E−04 | 4.3070 | 4.1184 | 0.7556 | 3.0603 |
| SIRT6 | Sirtuin (silent mating type | 1.29E−03 | 4.1209 | 3.8347 | 1.2244 | 3.0600 |
| | information regulation 2 homolog) 6 | | | | | |
| | (S. cerevisiae) | | | | | |
| HGF | Hepatocyte growth factor | 2.79E−02 | 0.8394 | 1.1850 | 7.1346 | 3.0530 |
| | (hepapoietin A; scatter factor) | | | | | |
| SYNE2 | Spectrin repeat containing | 2.71E−03 | 4.4489 | 4.0077 | 0.6917 | 3.0494 |
| DUSP1 | Dual specificity phosphatase 1 | 7.57E−03 | 3.5437 | 4.2084 | 1.2998 | 3.0173 |
| C10orf99 | Chromosome 10 open reading frame | 3.00E−02 | 1.7705 | 1.3690 | 5.8971 | 3.0122 |
| | 99 | | | | | |
| TMEM162 | Transmembrane protein 162 | 1.94E−03 | 4.3755 | 4.0055 | 0.6384 | 3.0064 |
| CGREF1 | Cell growth regulator with EF-hand | 9.92E−04 | 4.3379 | 4.0729 | 0.6024 | 3.0044 |
| | domain 1 | | | | | |
| ACY3 | Aspartoacylase (aminocyclase) 3 | 2.37E−03 | 4.3264 | 3.9244 | 0.7491 | 2.9999 |
| TLK2 | Tousled-like kinase 2 | 6.37E−05 | 4.2112 | 4.1446 | 0.6021 | 2.9860 |
| FLJ40432 | Hypothetical protein FLJ40432 | 2.40E−03 | 4.1812 | 3.7898 | 0.9617 | 2.9775 |
| SLC9A1 | Solute carrier family 9 | 1.64E−03 | 4.0955 | 3.7763 | 1.0274 | 2.9664 |
| | (sodium/hydrogen exchanger) | | | | | |
| C3orf60 | Chromosome 3 open reading frame | 1.12E−03 | 3.9194 | 3.6650 | 1.3145 | 2.9663 |
| | 60 | | | | | |
| PLEKHB1 | Pleckstrin homology domain | 3.84E−03 | 4.3865 | 3.8729 | 0.6364 | 2.9653 |
| | containing | | | | | |
| MICB | MHC class I polypeptide-related | 2.59E−02 | 3.9140 | 2.4397 | 2.5311 | 2.9616 |
| | sequence B | | | | | |
| KIAA1217 | KIAA1217 | 2.94E−05 | 3.9194 | 3.9621 | 0.9820 | 2.9545 |
| LOC339778 | Hypothetical protein LOC339778 | 3.68E−03 | 2.4802 | 2.8015 | 3.5455 | 2.9424 |
| HIF1AN | Hypoxia-inducible factor 1 | 1.22E−03 | 4.2006 | 3.9172 | 0.6506 | 2.9228 |
| TBX4 | T-box 4 | 1.25E−02 | 1.7172 | 1.3690 | 5.5877 | 2.8913 |
| ABO | ABO blood group (transferase A | 1.24E−02 | 4.4402 | 3.5420 | 0.6627 | 2.8816 |
| C16orf50 | Chromosome 16 open reading frame | 6.81E−05 | 3.7997 | 3.7375 | 1.1003 | 2.8792 |
| | 50 | | | | | |
| SVH | SVH protein | 7.78E−03 | 4.2672 | 3.5715 | 0.7982 | 2.8790 |
| LHX1 | LIM homeobox 1 | 6.51E−05 | 1.4473 | 1.4709 | 5.7113 | 2.8765 |
| LOC392617 | Similar to slit homolog 1 | 2.42E−03 | 4.0380 | 3.6590 | 0.9165 | 2.8711 |
| FLJ31222 | FLJ31222 protein | 2.43E−04 | 3.5616 | 3.4521 | 1.5932 | 2.8690 |
| GDA | Guanine deaminase | 4.02E−02 | 4.2134 | 2.7698 | 1.5640 | 2.8490 |
| PER2 | Period homolog 2 (Drosophila) | 7.72E−03 | 1.1817 | 1.4108 | 5.9319 | 2.8415 |
| TTN | Titin | 7.79E−03 | 3.1397 | 3.7515 | 1.6106 | 2.8339 |
| MUC4 | Mucin 4 | 6.17E−03 | 2.7182 | 2.3206 | 3.4524 | 2.8304 |
| RPL18A | Ribosomal protein L18a | 2.92E−02 | 4.3606 | 3.0640 | 1.0160 | 2.8135 |
| ASGR2 | Asialoglycoprotein receptor 2 | 2.28E−02 | 4.5663 | 2.9783 | 0.8056 | 2.7834 |
| JPH4 | Junctophilin 4 | 1.78E−02 | 3.2167 | 3.6769 | 1.3994 | 2.7643 |
| C3orf35 | Chromosome 3 open reading frame | 7.47E−03 | 1.3806 | 1.1598 | 5.6911 | 2.7438 |
| | 35 | | | | | |
| PRKAR1B | Protein kinase | 9.28E−04 | 3.8467 | 3.6192 | 0.7395 | 2.7351 |
| CYP11B1 | Cytochrome P450 | 1.01E−05 | 2.7330 | 2.7504 | 2.6821 | 2.7218 |
| INADL | InaD-like (Drosophila) | 2.97E−05 | 3.6899 | 3.6499 | 0.8139 | 2.7179 |
| LOC284998 | Hypothetical protein LOC284998 | 1.45E−04 | 3.0918 | 3.0181 | 2.0220 | 2.7106 |
| THSD1 | Thrombospondin | 1.01E−02 | 3.7286 | 3.4840 | 0.8133 | 2.6753 |
| TTC13 | Tetratricopeptide repeat domain 13 | 3.12E−02 | 4.1703 | 2.6585 | 1.1360 | 2.6549 |
| TMEM142A | Transmembrane protein 142A | 2.51E−02 | 2.3457 | 3.2487 | 2.3646 | 2.6530 |
| ATF3 | Activating transcription factor 3 | 3.72E−05 | 2.0334 | 2.0088 | 3.8940 | 2.6454 |
| LOC653073 | Similar to golgi autoantigen | 5.00E−04 | 4.3070 | 4.1184 | 0.7556 | 3.0603 |
| SIRT6 | Sirtuin (silent mating type | 1.29E−03 | 4.1209 | 3.8347 | 1.2244 | 3.0600 |
| | information regulation 2 homolog) 6 | | | | | |
| | (S. cerevisiae) | | | | | |
| HGF | Hepatocyte growth factor | 2.79E−02 | 0.8394 | 1.1850 | 7.1346 | 3.0530 |
| | (hepapoietin A; scatter factor) | | | | | |
| SYNE2 | Spectrin repeat containing | 2.71E−03 | 4.4489 | 4.0077 | 0.6917 | 3.0494 |
| DUSP1 | Dual specificity phosphatase 1 | 7.57E−03 | 3.5437 | 4.2084 | 1.2998 | 3.0173 |
| C10orf99 | Chromosome 10 open reading frame | 3.00E−02 | 1.7705 | 1.3690 | 5.8971 | 3.0122 |
| | 99 | | | | | |
| TMEM162 | Transmembrane protein 162 | 1.94E−03 | 4.3755 | 4.0055 | 0.6384 | 3.0064 |
| CGREF1 | Cell growth regulator with EF-hand | 9.92E−04 | 4.3379 | 4.0729 | 0.6024 | 3.0044 |
| | domain 1 | | | | | |
| ACY3 | Aspartoacylase (aminocyclase) 3 | 2.37E−03 | 4.3264 | 3.9244 | 0.7491 | 2.9999 |
| TLK2 | Tousled-like kinase 2 | 6.37E−05 | 4.2112 | 4.1446 | 0.6021 | 2.9860 |
| FLJ40432 | Hypothetical protein FLJ40432 | 2.40E−03 | 4.1812 | 3.7898 | 0.9617 | 2.9775 |
| SLC9A1 | Solute carrier family 9 | 1.64E−03 | 4.0955 | 3.7763 | 1.0274 | 2.9664 |
| | (sodium/hydrogen exchanger) | | | | | |
| C3orf60 | Chromosome 3 open reading frame | 1.12E−03 | 3.9194 | 3.6650 | 1.3145 | 2.9663 |
| | 60 | | | | | |
| PLEKHB1 | Pleckstrin homology domain | 3.84E−03 | 4.3865 | 3.8729 | 0.6364 | 2.9653 |
| | containing | | | | | |
| MICB | MHC class I polypeptide-related | 2.59E−02 | 3.9140 | 2.4397 | 2.5311 | 2.9616 |
| | sequence B | | | | | |
| KIAA1217 | KIAA1217 | 2.94E−05 | 3.9194 | 3.9621 | 0.9820 | 2.9545 |
| LOC339778 | Hypothetical protein LOC339778 | 3.68E−03 | 2.4802 | 2.8015 | 3.5455 | 2.9424 |
| HIF1AN | Hypoxia-inducible factor 1 | 1.22E−03 | 4.2006 | 3.9172 | 0.6506 | 2.9228 |
| TBX4 | T-box 4 | 1.25E−02 | 1.7172 | 1.3690 | 5.5877 | 2.8913 |
| ABO | ABO blood group (transferase A | 1.24E−02 | 4.4402 | 3.5420 | 0.6627 | 2.8816 |
| C16orf50 | Chromosome 16 open reading frame | 6.81E−05 | 3.7997 | 3.7375 | 1.1003 | 2.8792 |
| | 50 | | | | | |
| SVH | SVH protein | 7.78E−03 | 4.2672 | 3.5715 | 0.7982 | 2.8790 |
| LHX1 | LIM homeobox 1 | 6.51E−05 | 1.4473 | 1.4709 | 5.7113 | 2.8765 |
| LOC392617 | Similar to slit homolog 1 | 2.42E−03 | 4.0380 | 3.6590 | 0.9165 | 2.8711 |
| FLJ31222 | FLJ31222 protein | 2.43E−04 | 3.5616 | 3.4521 | 1.5932 | 2.8690 |
| GDA | Guanine deaminase | 4.02E−02 | 4.2134 | 2.7698 | 1.5640 | 2.8490 |
| PER2 | Period homolog 2 (Drosophila) | 7.72E−03 | 1.1817 | 1.4108 | 5.9319 | 2.8415 |
| TTN | Titin | 7.79E−03 | 3.1397 | 3.7515 | 1.6106 | 2.8339 |
| MUC4 | Mucin 4 | 6.17E−03 | 2.7182 | 2.3206 | 3.4524 | 2.8304 |
| RPL18A | Ribosomal protein L18a | 2.92E−02 | 4.3606 | 3.0640 | 1.0160 | 2.8135 |
| ASGR2 | Asialoglycoprotein receptor 2 | 2.28E−02 | 4.5663 | 2.9783 | 0.8056 | 2.7834 |
| JPH4 | Junctophilin 4 | 1.78E−02 | 3.2167 | 3.6769 | 1.3994 | 2.7643 |
| C3orf35 | Chromosome 3 open reading frame | 7.47E−03 | 1.3806 | 1.1598 | 5.6911 | 2.7438 |
| | 35 | | | | | |
| PRKAR1B | Protein kinase | 9.28E−04 | 3.8467 | 3.6192 | 0.7395 | 2.7351 |
| CYP11B1 | Cytochrome P450 | 1.01E−05 | 2.7330 | 2.7504 | 2.6821 | 2.7218 |
| INADL | InaD-like (Drosophila) | 2.97E−05 | 3.6899 | 3.6499 | 0.8139 | 2.7179 |
| LOC284998 | Hypothetical protein LOC284998 | 1.45E−04 | 3.0918 | 3.0181 | 2.0220 | 2.7106 |
| THSD1 | Thrombospondin | 1.01E−02 | 3.7286 | 3.4840 | 0.8133 | 2.6753 |
| TTC13 | Tetratricopeptide repeat domain 13 | 3.12E−02 | 4.1703 | 2.6585 | 1.1360 | 2.6549 |
| TMEM142A | Transmembrane protein 142A | 2.51E−02 | 2.3457 | 3.2487 | 2.3646 | 2.6530 |
| ATF3 | Activating transcription factor 3 | 3.72E−05 | 2.0334 | 2.0088 | 3.8940 | 2.6454 |
| SYNE2 | Spectrin repeat containing | 1.86E−02 | 4.0551 | 3.0690 | 0.8034 | 2.6425 |
| BZRAP1 | Benzodiazapine receptor (peripheral) | 3.32E−03 | 3.6800 | 3.2783 | 0.9081 | 2.6221 |
| | associated protein 1 | | | | | |
| SNX3 | Sorting nexin 3 | 8.16E−03 | 3.0020 | 3.7817 | 1.0734 | 2.6190 |
| FAM22A | Family with sequence similarity 22 | 4.42E−02 | 2.9739 | 4.0792 | 0.7860 | 2.6130 |
| SLC25A34 | Solute carrier family 25 | 6.24E−04 | 2.5071 | 2.3849 | 2.9446 | 2.6122 |
| MFI2 | Antigen p97 (melanoma associated) | 3.30E−02 | 2.4892 | 3.6285 | 1.6220 | 2.5799 |
| | identified by monoclonal antibodies | | | | | |
| | 133.2 and 96.5 | | | | | |
| UBE1L | Ubiquitin-activating enzyme E1-like | 5.70E−03 | 3.2541 | 2.7953 | 1.6845 | 2.5779 |
| RAP1GAP | RAP1 GTPase activating protein | 8.78E−03 | 3.1757 | 3.8373 | 0.6972 | 2.5700 |
| EGR1 | Early growth response 1 | 6.47E−03 | 2.3940 | 2.0934 | 3.2173 | 2.5682 |
| SSBP4 | Single stranded DNA binding protein 4 | 1.35E−02 | 2.4619 | 1.9455 | 3.2068 | 2.5381 |
| C9orf3 | Chromosome 9 open reading frame 3 | 3.59E−02 | 3.8205 | 2.5743 | 1.1141 | 2.5029 |
| FBXO21 | F-box protein 21 | 1.19E−03 | 3.4860 | 3.2530 | 0.6814 | 2.4734 |
| ADRA1B | Adrenergic | 4.56E−04 | 2.2937 | 2.3938 | 2.7132 | 2.4669 |
| IL31RA | Interleukin 31 receptor A | 3.29E−02 | 3.7508 | 2.5743 | 1.0744 | 2.4665 |
| NARF | Nuclear prelamin A recognition | 3.03E−02 | 3.1413 | 3.5082 | 0.7307 | 2.4601 |
| | factor | | | | | |
| PIK3R3 | Phosphoinositide-3-kinase | 4.66E−03 | 2.6727 | 2.3300 | 2.3750 | 2.4592 |
| DPH1 | DPH1 homolog (S. cerevisiae) | 2.58E−02 | 3.4503 | 2.4793 | 1.4470 | 2.4589 |
| KCNQ3 | Potassium voltage-gated channel | 1.48E−02 | 2.9915 | 3.5184 | 0.8119 | 2.4406 |
| DYSFIP1 | Dysferlin interacting protein 1 | 3.57E−02 | 3.7094 | 2.5038 | 1.1020 | 2.4384 |
| | (toonin) | | | | | |
| EFCAB2 | EF-hand calcium binding domain 2 | 4.92E−03 | 1.0073 | 1.1598 | 5.1372 | 2.4348 |
| CDKN2B | Cyclin-dependent kinase inhibitor 2B | 9.36E−05 | 3.3799 | 3.3152 | 0.6035 | 2.4329 |
| | (p15 | | | | | |
| GPR180 | G protein-coupled receptor 180 | 4.55E−02 | 2.0067 | 1.2806 | 3.9466 | 2.4113 |
| UPF3A | UPF3 regulator of nonsense | 1.67E−04 | 3.0918 | 3.0129 | 1.1195 | 2.4081 |
| | transcripts homolog A (yeast) | | | | | |
| STAB1 | Stabilin 1 | 7.21E−06 | 3.2445 | 3.2272 | 0.7466 | 2.4061 |
| CHIT1 | Chitinase 1 (chitotriosidase) | 1.80E−03 | 3.0972 | 2.8446 | 1.2463 | 2.3960 |
| HCN4 | Hyperpolarization activated cyclic | | | | | |
| | nucleotide-gated potassium channel 4 | 2.82E−03 | 1.6998 | 1.5280 | 3.8201 | 2.3493 |
| KIAA0415 | KIAA0415 protein | 8.59E−03 | 2.8451 | 3.4304 | 0.7123 | 2.3292 |
| SLC26A10 | Solute carrier family 26 | 1.51E−02 | 3.4382 | 2.7797 | 0.6600 | 2.2927 |
| C2orf17 | Chromosome 2 open | 4.58E−04 | 3.0304 | 2.9034 | 0.8222 | 2.2520 |
| | reading frame 17 | | | | | |
| SGCA | Sarcoglycan | 1.47E−03 | 2.1707 | 1.9545 | 2.6266 | 2.2506 |
| PIK3R1 | Phosphoinositide-3-kinase | 3.58E−02 | 2.2830 | 3.3856 | 1.0790 | 2.2492 |
| PDE4A | Phosphodiesterase 4A | 9.02E−03 | 3.1002 | 2.6398 | 0.8425 | 2.1942 |
| EBP | Emopamil binding protein | 2.12E−02 | 3.2729 | 2.4288 | 0.8345 | 2.1787 |
| | (sterol isomerase) | | | | | |
| IGJ | Immunoglobulin J | 1.54E−03 | 2.7762 | 2.5666 | 1.1622 | 2.1684 |
| | polypeptide | | | | | |
| RAB6IP2 | RAB6 interacting protein 2 | 3.38E−04 | 2.1940 | 2.1147 | 2.1919 | 2.1669 |
| KRTAP10-8 | Keratin associated protein | 1.07E−03 | 2.2937 | 2.1480 | 2.0474 | 2.1630 |
| | 10-8 | | | | | |
| COL7A1 | Collagen | 1.54E−02 | 2.3955 | 3.0838 | 0.9935 | 2.1576 |
| CYorf16 | Chromosome Y open | 9.10E−04 | 2.2282 | 2.3669 | 1.7970 | 2.1307 |
| | reading frame 16 | | | | | |
| IFNAR2 | Interferon (alpha | 1.20E−02 | 2.1226 | 1.7004 | 2.5604 | 2.1278 |
| NKPD1 | NTPase | 2.84E−02 | 1.1544 | 1.6345 | 3.5863 | 2.1250 |
| PTGER1 | Prostaglandin E receptor 1 | 8.73E−03 | 2.8584 | 2.3669 | 1.1264 | 2.1172 |
| | (subtype EP1) | | | | | |
| CAMK1D | Calcium/calmodulin- | 2.87E−02 | 2.6254 | 1.8507 | 1.8708 | 2.1156 |
| | dependent protein kinase | | | | | |
| | ID | | | | | |
| C9orf138 | Chromosome 9 open | 1.77E−02 | 1.7679 | 1.3475 | 3.2192 | 2.1115 |
| | reading frame 138 | | | | | |
| LOC440669 | Hypothetical LOC440669 | 5.69E−03 | 2.7548 | 2.3669 | 1.1878 | 2.1032 |
| SYNE2 | Spectrin repeat containing | 4.55E−02 | 2.1940 | 3.4377 | 0.6629 | 2.0982 |
| LOC389844 | Similar to ferritin | 1.97E−03 | 2.2614 | 2.0688 | 1.9632 | 2.0978 |
| PPP4R1 | Protein phosphatase 4 | 2.36E−02 | 2.0201 | 2.7698 | 1.4929 | 2.0943 |
| AIF1 | Allograft inflammatory | 5.87E−04 | 2.8451 | 2.7104 | 0.6901 | 2.0819 |
| | factor 1 | | | | | |
| USP41 | Ubiquitin specific peptidase | 1.03E−02 | 3.0247 | 2.4627 | 0.7487 | 2.0787 |
| | 41 | | | | | |
| C1orf182 | Chromosome 1 open | 1.93E−02 | 2.9658 | 2.2324 | 0.9856 | 2.0612 |
| | reading frame 182 | | | | | |
| ANK3 | Ankyrin 3 | 5.24E−04 | 2.1707 | 2.2724 | 1.6916 | 2.0449 |
| STAT5B | Signal transducer and | 1.61E−03 | 2.1588 | 2.3394 | 1.6300 | 2.0427 |
| | activator of transcription 5B | | | | | |
| SNAP23 | Synaptosomal-associated | 5.10E−04 | 1.9654 | 1.8786 | 2.2800 | 2.0413 |
| | protein | | | | | |
| PCDH7 | BH-protocadherin (brain- | 1.58E−02 | 1.7679 | 1.3690 | 2.9850 | 2.0406 |
| | heart) | | | | | |
| ZADH1 | Zinc binding alcohol | 7.56E−04 | 1.4688 | 1.3901 | 3.2143 | 2.0244 |
| | dehydrogenase | | | | | |
| KIFC3 | Kinesin family member C3 | 3.83E−02 | 2.0596 | 1.3690 | 2.6314 | 2.0200 |
| TRIM16 | Tripartite motif-containing | 8.28E−04 | 2.2282 | 2.1034 | 1.6826 | 2.0047 |
| | 16 | | | | | |
| GPR142 | G protein-coupled receptor | 4.48E−03 | 2.0201 | 2.3111 | 1.6824 | 2.0046 |
| | 142 | | | | | |
| CBWD1 | COBW domain containing 1 | 3.75E−02 | 3.1501 | 2.1034 | 0.7185 | 1.9907 |
| PPP1R3G | Protein phosphatase 1 | 1.04E−03 | 2.4244 | 2.2724 | 1.2299 | 1.9756 |
| LRRK1 | Leucine-rich repeat kinase 1 | 5.30E−04 | 2.5160 | 2.4026 | 0.9370 | 1.9519 |
| AFF1 | AF4/FMR2 family | 3.30E−02 | 0.9094 | 1.3256 | 3.6069 | 1.9473 |
| PRSS36 | Protease | 4.60E−03 | 1.9654 | 2.2526 | 1.6237 | 1.9472 |
| SMC1B | SMC1 structural | 1.87E−07 | 1.8631 | 1.8647 | 2.1050 | 1.9443 |
| | maintenance of | | | | | |
| | chromosomes 1-like 2 | | | | | |
| | (yeast) | | | | | |
| HS1BP3 | HS1-binding protein 3 | 1.84E−02 | 2.9164 | 2.2119 | 0.6235 | 1.9173 |
| AGER | Advanced glycosylation end | 4.89E−02 | 1.5899 | 0.9962 | 3.1435 | 1.9099 |
| | product-specific receptor | | | | | |
| LOC340281 | Hypothetical protein | 4.70E−04 | 2.4052 | 2.5118 | 0.8097 | 1.9089 |
| | LOC340281 | | | | | |
| SFTPA1 | Surfactant | 3.14E−03 | 1.8478 | 1.6513 | 2.1979 | 1.8990 |
| TEX13B | Testis expressed sequence | 3.71E−02 | 3.0015 | 2.0088 | 0.6794 | 1.8966 |
| | 13B | | | | | |
| PSPN | Persephin | 4.21E−03 | 2.1469 | 2.4458 | 1.0816 | 1.8914 |
| SPTY2D1 | SPT2 | 3.18E−03 | 2.6092 | 2.3300 | 0.7039 | 1.8811 |
| LOC124216 | Hypothetical LOC124216 | 4.95E−04 | 2.3148 | 2.4201 | 0.9005 | 1.8784 |
| TUBA4 | Tubulin | 1.81E−02 | 2.5421 | 1.9324 | 1.1410 | 1.8719 |
| GCK | Glucokinase (hexokinase 4 | 1.21E−03 | 2.4148 | 2.2526 | 0.9400 | 1.8691 |
| FGF6 | Fibroblast growth factor 6 | 5.27E−03 | 2.4982 | 2.1589 | 0.9468 | 1.8680 |
| TMEM111 | Transmembrane protein | 3.03E−02 | 1.3806 | 1.5735 | 2.6214 | 1.8585 |
| | 111 | | | | | |
| TRPM4 | Transient receptor potential | 4.69E−02 | 1.3806 | 2.1804 | 2.0069 | 1.8559 |
| | cation channel | | | | | |
| FLJ22531 | Hypothetical protein | 6.27E−03 | 2.4802 | 2.1147 | 0.9672 | 1.8540 |
| | FLJ22531 | | | | | |
| FLJ36116 | Hypothetical locus | 1.79E−02 | 2.7330 | 2.0805 | 0.7356 | 1.8497 |
| | LOC388666 | | | | | |
| RANBP6 | RAN binding protein 6 | 1.62E−02 | 1.7172 | 1.3256 | 2.4960 | 1.8463 |
| CLPS | Colipase | 5.87E−03 | 1.6461 | 1.4108 | 2.4375 | 1.8315 |
| CEP152 | Centrosomal protein | 1.40E−02 | 2.5160 | 2.3531 | 0.6209 | 1.8300 |
| | 152 kDa | | | | | |
| PLCXD1 | Phosphatidylinositol- | 2.15E−03 | 2.1348 | 1.9455 | 1.3982 | 1.8261 |
| | specific phospholipase C | | | | | |
| DIP2A | DIP2 disco-interacting | 1.52E−03 | 2.0067 | 2.1697 | 1.2815 | 1.8193 |
| | protein 2 homolog A | | | | | |
| | (Drosophila) | | | | | |
| ARPP-21 | Cyclic AMP-regulated | 2.66E−03 | 2.1824 | 2.4201 | 0.8436 | 1.8154 |
| | phosphoprotein | | | | | |
| PER2 | Period homolog 2 | 1.54E−04 | 2.0067 | 2.0571 | 1.3740 | 1.8126 |
| | (Drosophila) | | | | | |
| ANGPTL4 | Angiopoietin-like 4 | 2.62E−02 | 2.1588 | 1.5464 | 1.7158 | 1.8070 |
| RETN | Resistin | 1.73E−03 | 2.2504 | 2.4458 | 0.7045 | 1.8002 |
| MSH5 | MutS homolog 5 (E. coli) | 6.22E−03 | 1.8783 | 2.2015 | 1.3019 | 1.7939 |
| LOC653224 | Similar to F-box only | 1.49E−02 | 2.3355 | 1.8222 | 1.2222 | 1.7933 |
| | protein 25 isoform 2 | | | | | |
| FLJ25778 | Hypothetical protein | 2.44E−03 | 2.1102 | 2.3300 | 0.9367 | 1.7923 |
| | FLJ25778 | | | | | |
| MGC4172 | Short-chain | 4.30E−04 | 2.1707 | 2.2626 | 0.9362 | 1.7898 |
| | dehydrogenase/reductase | | | | | |
| LOC388796 | Hypothetical LOC388796 | 5.15E−03 | 2.3087 | 2.0938 | 0.8362 | 1.7462 |
| SYNE2 | Spectrin repeat containing | 1.16E−03 | 1.5105 | 1.4108 | 2.2806 | 1.7340 |
| FOXR2 | Forkhead box R2 | 2.16E−02 | 1.6752 | 1.9451 | 1.5725 | 1.7309 |
| IER2 | Immediate early response 2 | 5.97E−03 | 1.9271 | 1.5572 | 1.6843 | 1.7228 |
| TBC1D10A | TBC1 domain family | 1.61E−02 | 2.4130 | 1.6725 | 1.0806 | 1.7220 |
| SH3PX3 | SH3 and PX domain | 6.26E−04 | 2.0724 | 1.9712 | 1.1221 | 1.7219 |
| | containing 3 | | | | | |
| SIM2 | Single-minded homolog 2 | 6.45E−05 | 1.5899 | 1.5646 | 1.9964 | 1.7170 |
| | (Drosophila) | | | | | |
| ANKRD26 | Ankyrin repeat domain 26 | 2.19E−02 | 2.3251 | 1.7164 | 1.0906 | 1.7107 |
| PDLIM4 | PDZ and LIM domain 4 | 5.33E−03 | 2.2830 | 1.9712 | 0.8731 | 1.7091 |
| RUTBC3 | RUN and TBC1 domain | 5.20E−03 | 2.4052 | 2.0805 | 0.6227 | 1.7028 |
| | containing 3 | | | | | |
| SPAG6 | Sperm associated antigen 6 | 3.72E−02 | 2.6804 | 1.7929 | 0.6136 | 1.6956 |
| CA7 | Carbonic anhydrase VII | 1.13E−02 | 2.2055 | 1.7780 | 1.0908 | 1.6914 |
| MFI2 | Antigen p97 (melanoma | 9.69E−03 | 2.2055 | 1.8076 | 1.0236 | 1.6789 |
| | associated) identified by | | | | | |
| | monoclonal antibodies | | | | | |
| | 133.2 and 96.5 | | | | | |
| TMPRSS3 | Transmembrane protease | 3.56E−05 | 1.6643 | 1.6843 | 1.6280 | 1.6588 |
| FLJ35390 | Hypothetical protein | 6.08E−03 | 1.9513 | 1.6679 | 1.3536 | 1.6576 |
| | FLJ35390 | | | | | |
| THAP6 | THAP domain containing 6 | 3.38E−03 | 1.7172 | 1.5280 | 1.6869 | 1.6440 |
| HOMER2 | Homer homolog 2 | 3.67E−02 | 2.4619 | 1.6513 | 0.8103 | 1.6411 |
| | (Drosophila) | | | | | |
| H1FOO | H1 histone family | 2.19E−02 | 1.9654 | 1.4512 | 1.4919 | 1.6361 |
| KIAA1443 | KIAA1443 | 2.59E−02 | 2.1940 | 1.3691 | 1.3423 | 1.6351 |
| HMGCR | 3-hydroxy-3-methylglutaryl- | 3.09E−04 | 1.8783 | 1.9455 | 1.0749 | 1.6329 |
| | Coenzyme A reductase | | | | | |
| LOC345630 | Similar to fibrillarin | 4.16E−02 | 1.3339 | 2.0452 | 1.4441 | 1.6077 |
| S100A11 | S100 calcium binding | 3.42E−02 | 2.2169 | 1.5092 | 1.0827 | 1.6030 |
| | protein A11 (calgizzarin) | | | | | |
| RAB35 | RAB35 | 5.35E−03 | 2.0067 | 1.7321 | 1.0640 | 1.6009 |
| CARD14 | Caspase recruitment | 6.75E−03 | 2.2169 | 1.8786 | 0.6445 | 1.5800 |
| | domain family | | | | | |
| CCDC59 | Coiled-coil domain | 4.07E−04 | 1.8932 | 1.9712 | 0.8510 | 1.5718 |
| | containing 59 | | | | | |
| CRTC2 | CREB regulated | 4.25E−02 | 1.2442 | 1.5554 | 1.9102 | 1.5700 |
| | transcription coactivator 2 | | | | | |
| PSMA1 | Proteasome (prosome | 1.58E−03 | 2.1348 | 1.9712 | 0.6029 | 1.5696 |
| COL9A1 | Collagen | 2.28E−02 | 1.9226 | 1.4108 | 1.3643 | 1.5659 |
| OR2Y1 | Olfactory receptor | 1.40E−02 | 2.2614 | 1.7780 | 0.6432 | 1.5609 |
| PPP1R12C | Protein phosphatase 1 | 1.78E−02 | 2.1226 | 1.6174 | 0.9390 | 1.5596 |
| CARD14 | Caspase recruitment | 7.99E−04 | 2.0724 | 1.9584 | 0.6447 | 1.5585 |
| | domain family | | | | | |
| REXO4 | REX4 | 3.32E−02 | 1.8005 | 1.2338 | 1.6346 | 1.5563 |
| ANP32D | Acidic (leucine-rich) nuclear | 4.88E−04 | 2.0334 | 1.9455 | 0.6893 | 1.5561 |
| | phosphoprotein 32 family | | | | | |
| RPS3 | Ribosomal protein S3 | 9.13E−04 | 1.6998 | 1.6000 | 1.3597 | 1.5532 |
| ZNF83 | Zinc finger protein 83 | 1.65E−02 | 2.1226 | 1.6345 | 0.8799 | 1.5456 |
| C14orf78 | Chromosome 14 open | 1.38E−04 | 1.7512 | 1.7929 | 1.0311 | 1.5250 |
| | reading frame 78 | | | | | |
| REXO1L2P | REX1 | 2.00E−03 | 1.9931 | 1.8222 | 0.7418 | 1.5190 |
| PDCD4 | Programmed cell death 4 | 9.54E−03 | 1.7679 | 1.4512 | 1.3344 | 1.5178 |
| | (neoplastic transformation | | | | | |
| | inhibitor) | | | | | |
| IL31 | Interleukin 31 | 1.41E−03 | 1.8631 | 2.0088 | 0.6692 | 1.5137 |
| BSND | Bartter syndrome | 8.73E−03 | 1.7512 | 2.1147 | 0.6557 | 1.5072 |
| FLJ21736 | Esterase 31 | 2.24E−04 | 1.8164 | 1.7629 | 0.9330 | 1.5041 |
| EGFL9 | EGF-like-domain | 2.30E−02 | 2.0596 | 1.5092 | 0.9426 | 1.5038 |
| KNDC1 | Kinase non-catalytic C-lobe | 1.75E−02 | 2.2055 | 1.6843 | 0.6186 | 1.5028 |
| | domain (KIND) containing 1 | | | | | |
| NMT2 | N-myristoyltransferase 2 | 4.48E−02 | 2.1707 | 1.3901 | 0.9333 | 1.4980 |
| PCDH1 | Protocadherin 1 (cadherin- | 2.21E−02 | 2.1226 | 1.5646 | 0.8064 | 1.4978 |
| | like 1) | | | | | |
| C1orf201 | Chromosome 1 open | 4.01E−03 | 1.2602 | 1.4312 | 1.7863 | 1.4926 |
| | reading frame 201 | | | | | |
| C1orf116 | Chromosome 1 open | 1.92E−04 | 1.6998 | 1.7476 | 1.0300 | 1.4925 |
| | reading frame 116 | | | | | |
| FLJ35348 | FLJ35348 | 9.67E−03 | 1.5899 | 1.3033 | 1.5826 | 1.4920 |
| BTBD6 | BTB (POZ) domain | 5.37E−04 | 1.8322 | 1.9192 | 0.7186 | 1.4900 |
| | containing 6 | | | | | |
| DKFZP434O047 | DKFZP434O047 protein | 1.63E−02 | 1.9080 | 1.4709 | 1.0532 | 1.4773 |
| KCNN4 | Potassium | 1.20E−02 | 2.1226 | 1.7004 | 0.6059 | 1.4763 |
| | intermediate/small | | | | | |
| | conductance calcium- | | | | | |
| | activated channel | | | | | |
| LPIN2 | Lipin 2 | 7.88E−03 | 1.3806 | 1.6513 | 1.3683 | 1.4667 |
| MEGF11 | Multiple EGF-like-domains | 1.83E−02 | 2.0852 | 1.5824 | 0.7280 | 1.4652 |
| | 11 | | | | | |
| HTR3E | 5-hydroxytryptamine | 2.72E−02 | 1.6998 | 1.2096 | 1.4817 | 1.4637 |
| | (serotonin) receptor 3 | | | | | |
| TEAD4 | TEA domain family | 8.30E−03 | 1.7679 | 1.4709 | 1.1292 | 1.4560 |
| | member 4 | | | | | |
| PQBP1 | Polyglutamine binding | 1.61E−02 | 1.4688 | 1.1341 | 1.7079 | 1.4369 |
| | protein 1 | | | | | |
| CARD14 | Caspase recruitment | 4.50E−02 | 1.5105 | 0.9666 | 1.8263 | 1.4345 |
| | domain family | | | | | |
| PPCDC | Phosphopantothenoyl- | 1.83E−02 | 1.8322 | 1.3901 | 1.0708 | 1.4310 |
| | cysteine decarboxylase | | | | | |
| CARD4 | Caspase recruitment | 3.64E−02 | 1.6089 | 1.0809 | 1.5958 | 1.4285 |
| | domain family | | | | | |
| KCNG2 | Potassium voltage-gated | 1.02E−02 | 1.3796 | 1.7603 | 1.1406 | 1.4269 |
| | channel | | | | | |
| C1orf25 | Chromosome 1 open | 3.83E−02 | 1.1817 | 1.7780 | 1.2991 | 1.4196 |
| | reading frame 25 | | | | | |
| LOC222159 | Hypothetical protein | 1.55E−02 | 1.4032 | 1.8076 | 1.0279 | 1.4129 |
| | LOC222159 | | | | | |
| ACHE | Acetylcholinesterase (Yt | 2.84E−03 | 1.4688 | 1.6345 | 1.0952 | 1.3995 |
| | blood group) | | | | | |
| C15orf20 | Chromosome 15 open | 2.11E−02 | 1.5105 | 2.0332 | 0.6411 | 1.3949 |
| | reading frame 20 | | | | | |
| SCUBE2 | Signal peptide | 1.87E−02 | 1.3339 | 1.7629 | 1.0744 | 1.3904 |
| FOXH1 | Forkhead box H1 | 1.89E−02 | 1.1817 | 1.5646 | 1.4218 | 1.3894 |
| EIF2B4 | Eukaryotic translation | 9.80E−03 | 1.6461 | 1.3475 | 1.0901 | 1.3613 |
| | initiation factor 2B | | | | | |
| PAX2 | Paired box gene 2 | 2.56E−02 | 1.3806 | 1.9192 | 0.7471 | 1.3489 |
| ZBTB39 | Zinc finger and BTB | 6.16E−03 | 1.5309 | 1.7929 | 0.7116 | 1.3451 |
| | domain containing 39 | | | | | |
| FGF22 | Fibroblast growth factor 22 | 1.61E−02 | 1.8783 | 1.4512 | 0.7021 | 1.3438 |
| SVIL | Supervillin | 8.77E−03 | 1.8005 | 1.4902 | 0.7005 | 1.3304 |
| CFB | Complement factor B | 6.26E−04 | 1.6822 | 1.6000 | 0.7088 | 1.3303 |
| KIAA0746 | KIAA0746 protein | 1.27E−02 | 1.4255 | 1.1341 | 1.4287 | 1.3294 |
| FLJ20309 | Hypothetical protein | 4.06E−04 | 1.0382 | 1.0809 | 1.8580 | 1.3257 |
| | FLJ20309 | | | | | |
| CD40 | CD40 molecule | 2.61E−03 | 1.3098 | 1.4512 | 1.1976 | 1.3195 |
| C9orf156 | Chromosome 9 open reading | 3.74E−02 | 1.9513 | 1.3033 | 0.6826 | 1.3124 |
| | frame 156 | | | | | |
| DPAGT1 | Dolichyl-phosphate (UDP-N- | 3.17E−02 | 1.7843 | 1.2338 | 0.9132 | 1.3104 |
| | acetylglucosamine) N-acetyl- | | | | | |
| | glucosamine-phosphotransferase | | | | | |
| | 1 (GlcNAc-1-P transferase) | | | | | |
| ACE | Angiotensin I converting | 4.76E−02 | 1.9931 | 1.2574 | 0.6754 | 1.3086 |
| | enzyme (peptidyl-dipeptidase A) 1 | | | | | |
| DRB1 | Developmentally regulated | 1.75E−02 | 1.2346 | 1.6174 | 1.0642 | 1.3054 |
| | RNA-binding protein 1 | | | | | |
| SLC15A3 | Solute carrier family 15 | 1.06E−02 | 1.8559 | 1.4431 | 0.6150 | 1.3047 |
| LOC283953 | Hypothetical LOC283953 | 3.46E−02 | 1.0382 | 1.5280 | 1.3183 | 1.2948 |
| BRWD1 | Bromodomain and WD repeat | 3.14E−05 | 1.4473 | 1.4312 | 0.9935 | 1.2907 |
| | domain containing 1 | | | | | |
| SEMA4A | Sema domain | 3.45E−02 | 1.2085 | 1.7780 | 0.8842 | 1.2902 |
| LOC283692 | Hypothetical protein | 8.27E−04 | 1.5105 | 1.6000 | 0.7533 | 1.2880 |
| | LOC283692 | | | | | |
| FHAD1 | Forkhead-associated (FHA) | 1.16E−03 | 1.5105 | 1.6174 | 0.7104 | 1.2794 |
| | phosphopeptide binding domain 1 | | | | | |
| ABCD2 | ATP-binding cassette | 3.95E−03 | 1.4255 | 1.6174 | 0.7843 | 1.2757 |
| DMRTC2 | DMRT-like family C2 | 1.41E−02 | 0.8394 | 0.6595 | 2.3229 | 1.2739 |
| DHRS4 | Dehydrogenase/reductase (SDR | 3.68E−02 | 1.7679 | 1.1850 | 0.8390 | 1.2640 |
| | family) member 4 | | | | | |
| CDH4 | Cadherin 4 | 4.79E−02 | 1.6277 | 1.0251 | 1.1306 | 1.2611 |
| RNF128 | Ring finger protein 128 | 4.00E−02 | 1.8005 | 1.1850 | 0.7808 | 1.2554 |
| HMGCL | 3-hydroxymethyl-3- | 4.31E−03 | 1.4473 | 1.6513 | 0.6664 | 1.2550 |
| | methylglutaryl-Coenzyme A | | | | | |
| | lyase | | | | | |
| | (hydroxymethylglutaricaciduria) | | | | | |
| CLEC11A | C-type lectin domain family 11 | 4.84E−03 | 1.6461 | 1.4312 | 0.6444 | 1.2406 |
| ZAK | Sterile alpha motif and leucine | 3.20E−02 | 1.7172 | 1.1850 | 0.8072 | 1.2365 |
| | zipper containing kinase AZK | | | | | |
| VN1R1 | Vomeronasal 1 receptor 1 | 1.66E−02 | 1.7512 | 1.3475 | 0.6048 | 1.2345 |
| FOSL2 | FOS-like antigen 2 | 6.17E−06 | 1.3098 | 1.3033 | 1.0834 | 1.2322 |
| UBE4A | Ubiquitination factor E4A | 1.28E−02 | 1.2853 | 1.6174 | 0.7803 | 1.2276 |
| | (UFD2 homolog | | | | | |
| KCNMB1 | Potassium large conductance | 4.62E−02 | 1.7843 | 1.1341 | 0.7618 | 1.2267 |
| | calcium-activated channel | | | | | |
| PHKB | Phosphorylase kinase | 1.41E−03 | 0.9755 | 0.9050 | 1.7985 | 1.2263 |
| IRF8 | Interferon regulatory factor 8 | 1.10E−02 | 1.2236 | 0.9069 | 1.4957 | 1.2087 |
| 4-Sep | Septin 4 | 4.08E−03 | 1.1264 | 1.2806 | 1.1939 | 1.2003 |
| PTCD2 | Pentatricopeptide repeat domain 2 | 2.94E−03 | 1.4032 | 1.5646 | 0.6178 | 1.1952 |
| OR2B11 | Olfactory receptor | 1.18E−05 | 1.3806 | 1.3901 | 0.8128 | 1.1945 |
| DLEU8 | Deleted in lymphocytic leukemia 8 | 3.34E−03 | 1.3098 | 1.4709 | 0.7964 | 1.1924 |
| NFASC | Neurofascin homolog (chicken) | 7.34E−03 | 0.9094 | 1.0809 | 1.5700 | 1.1868 |
| CACNA2D1 | Calcium channel | 4.30E−02 | 1.4473 | 0.9362 | 1.1571 | 1.1802 |
| MGC21830 | Hypothetical protein | 1.75E−02 | 1.2085 | 1.5824 | 0.7353 | 1.1754 |
| | MGC21830 | | | | | |
| FLJ00038 | CXYorf1-related protein | 2.51E−03 | 1.4898 | 1.3475 | 0.6142 | 1.1505 |
| GBF1 | Golgi-specific brefeldin A | 9.60E−06 | 1.3339 | 1.3256 | 0.7894 | 1.1496 |
| | resistance factor 1 | | | | | |
| ZAP70 | Zeta-chain (TCR) associated | 8.90E−03 | 1.4032 | 1.1598 | 0.8683 | 1.1438 |
| | protein kinase 70 kDa | | | | | |
| GJA10 | Gap junction protein | ##### | 1.2346 | 1.2338 | 0.9560 | 1.1415 |
| PIGN | Phosphatidylinositol glycan | 3.67E−03 | 1.3098 | 1.1598 | 0.9046 | 1.1248 |
| SORL1 | Sortilin-related receptor | 1.84E−02 | 1.4255 | 1.0809 | 0.8435 | 1.1166 |
| ITLN1 | Intelectin 1 (galactofuranose | 7.72E−03 | 1.1817 | 1.4108 | 0.7358 | 1.1095 |
| | binding) | | | | | |
| OCRL | Oculocerebrorenal syndrome of | 6.43E−04 | 1.1264 | 1.1850 | 0.9865 | 1.0993 |
| | Lowe | | | | | |
| GRM4 | Glutamate receptor | 1.01E−02 | 1.3574 | 1.1078 | 0.8110 | 1.0921 |
| ZCCHC7 | Zinc finger | 1.00E−02 | 1.3843 | 1.0959 | 0.7770 | 1.0858 |
| SDS | Serine dehydratase | 4.15E−02 | 1.1817 | 0.7710 | 1.2797 | 1.0775 |
| TMEM88 | Transmembrane protein 88 | 3.74E−02 | 1.4473 | 0.9666 | 0.7913 | 1.0684 |
| VRK1 | Vaccinia related kinase 1 | 8.94E−03 | 0.9755 | 0.8060 | 1.2946 | 1.0254 |
| MAGED2 | Melanoma antigen family D | 2.07E−02 | 0.6005 | 0.8060 | 1.6452 | 1.0172 |
| PTGIR | Prostaglandin I2 (prostacyclin) | 2.21E−03 | 1.1264 | 1.0251 | 0.8770 | 1.0095 |
| | receptor (IP) | | | | | |
| MXD3 | MAX dimerization protein 3 | 3.61E−03 | 0.9094 | 0.8060 | 1.3094 | 1.0083 |
| C14orf172 | Chromosome 14 open reading | 3.96E−03 | 0.8749 | 0.7710 | 1.3683 | 1.0047 |
| | frame 172 | | | | | |
| OPTN | Optineurin | 1.68E−03 | 0.8749 | 0.8060 | 1.3324 | 1.0044 |
| ZNF740 | Zinc finger protein 740 | 4.57E−03 | 1.0977 | 1.2574 | 0.6561 | 1.0037 |
| PDLIM1 | PDZ and LIM domain 1 (elfin) | 1.01E−03 | 1.2085 | 1.1341 | 0.6312 | 0.9913 |
| CX40.1 | Connexin40.1 | 1.80E−03 | 0.8394 | 0.7710 | 1.3603 | 0.9902 |
| GAL3ST2 | Galactose-3-O-sulfotransferase 2 | 1.68E−03 | 0.8749 | 0.8060 | 1.2537 | 0.9782 |
| ASPH | Aspartate beta-hydroxylase | 2.64E−02 | 1.1264 | 0.8060 | 0.9289 | 0.9538 |
| ITM2A | Integral membrane protein 2A | 4.62E−02 | 1.0977 | 0.6978 | 1.0575 | 0.9510 |
| SCAND2 | SCAN domain containing 2 | 2.46E−02 | 0.8027 | 1.1078 | 0.9294 | 0.9466 |
| PVT1 | Pvt1 oncogene homolog | 4.23E−04 | 0.9755 | 0.9362 | 0.9132 | 0.9416 |
| FLJ32130 | Hypothetical protein FLJ32130 | 2.29E−02 | 1.2346 | 0.9050 | 0.6365 | 0.9254 |
| CCDC82 | Coiled-coil domain containing | 3.98E−04 | 0.7650 | 0.7350 | 1.2334 | 0.9111 |
| | 82 | | | | | |
| ADAMTS2 | ADAM metallopeptidase with | 2.42E−02 | 0.7650 | 1.0533 | 0.8440 | 0.8874 |
| | thrombospondin type 1 motif | | | | | |
| NME6 | Non-metastatic cells 6 | 4.14E−02 | 1.2346 | 0.8060 | 0.6214 | 0.8874 |
| RPS6KB1 | Ribosomal protein S6 kinase | 4.02E−02 | 0.9429 | 0.6198 | 1.0925 | 0.8851 |
| CRYBA2 | Crystallin | 7.17E−03 | 0.9656 | 0.9804 | 0.7015 | 0.8825 |
| SNAPC1 | Small nuclear RNA activating | 4.19E−04 | 0.9094 | 0.8729 | 0.8545 | 0.8789 |
| | complex | | | | | |
| WHSC1 | Wolf-Hirschhorn syndrome | 3.77E−04 | 0.6856 | 0.6595 | 1.2429 | 0.8627 |
| | candidate 1 | | | | | |
| B2M | Beta-2-microglobulin | 3.58E−02 | 0.7650 | 1.1341 | 0.6121 | 0.8371 |
| TRIM55 | Tripartite motif-containing 55 | 1.41E−02 | 0.6856 | 0.8729 | 0.9383 | 0.8323 |
| UBE1DC1 | Ubiquitin-activating enzyme E1- | 5.57E−03 | 0.6005 | 0.6978 | 1.0235 | 0.7740 |
| | domain containing 1 | | | | | |
| KLHL26 | Kelch-like 26 (Drosophila) | 3.96E−03 | 0.8749 | 0.7710 | 0.6000 | 0.7486 |
| RBM16 | RNA binding motif protein 16 | 3.85E−02 | 0.6005 | 0.9050 | 0.6097 | 0.7051 |
| KCNC2 | Potassium voltage-gated channel | 9.07E−04 | 0.7259 | 0.7710 | 0.6053 | 0.7008 |
| RPN2 | Ribophorin II | 4.49E−02 | −0.8494 | −0.7941 | −0.6118 | −0.7518 |
| YWHAZ | Tyrosine 3- | 1.95E−02 | −0.8323 | −0.7732 | −0.7855 | −0.7970 |
| | monooxygenase/tryptophan 5- | | | | | |
| | monooxygenase activation | | | | | |
| | protein | | | | | |
| CDC42EP3 | CDC42 effector protein (Rho | 3.86E−02 | −0.8604 | −0.9273 | −0.6397 | −0.8092 |
| | GTPase binding) 3 | | | | | |
| ZNF337 | Zinc finger protein 337 | 3.69E−02 | −0.9537 | −0.9669 | −0.6362 | −0.8523 |
| HSPA4 | Heat shock 70 kDa protein 4 | 3.45E−02 | −0.9664 | −0.8996 | −0.6994 | −0.8551 |
| ARL8A | ADP-ribosylation factor-like 8A | 3.34E−02 | −1.0240 | −1.0666 | −0.6132 | −0.9013 |
| RQCD1 | RCD1 required for cell | 3.17E−02 | −1.1217 | −0.9994 | −0.6527 | −0.9246 |
| | differentiation1 homolog (S. pombe) | | | | | |
| STRA13 | Stimulated by retinoic acid 13 | 3.64E−02 | −0.9665 | −1.0846 | −0.7775 | −0.9429 |
| | homolog (mouse) | | | | | |
| FPGS | Folylpolyglutamate synthase | 1.53E−02 | −1.0263 | −1.3332 | −0.6005 | −0.9867 |
| B2M | Beta-2-microglobulin | 1.45E−02 | −0.8661 | −1.1391 | −1.0117 | −1.0056 |
| TMED2 | Transmembrane emp24 domain | 4.87E−03 | −0.9639 | −1.0726 | −0.9863 | −1.0076 |
| | trafficking protein 2 | | | | | |
| NDFIP1 | Nedd4 family interacting protein 1 | 4.52E−02 | −0.8902 | −1.0237 | −1.1257 | −1.0132 |
| SP3 | Sp3 transcription factor | 2.44E−02 | −1.0598 | −1.1820 | −0.8000 | −1.0140 |
| LOC84661 | Dpy-30-like protein | 1.98E−03 | −0.7935 | −0.6995 | −1.5783 | −1.0238 |
| GTF2A2 | General transcription factor IIA | 3.17E−02 | −1.2640 | −1.2253 | −0.6036 | −1.0310 |
| NUDT21 | Nudix (nucleoside diphosphate | 4.92E−02 | −1.3819 | −1.0218 | −0.7010 | −1.0349 |
| | linked moiety X)-type motif 21 | | | | | |
| TNFRSF25 | Tumor necrosis factor receptor | 3.06E−02 | −1.2207 | −1.2191 | −0.6761 | −1.0386 |
| | superfamily | | | | | |
| STAM | Signal transducing adaptor | 1.33E−02 | −1.0125 | −1.3981 | −0.7198 | −1.0435 |
| | molecule (SH3 domain and | | | | | |
| | ITAM motif) 1 | | | | | |
| ARS2 | ARS2 protein | 3.96E−02 | −1.4616 | −0.8882 | −0.8076 | −1.0524 |
| HK1 | Hexokinase 1 | 3.73E−02 | −1.0520 | −1.2191 | −0.9449 | −1.0720 |
| ELF2 | E74-like factor 2 (ets domain | 3.99E−02 | −0.9659 | −1.1582 | −1.0934 | −1.0725 |
| | transcription factor) | | | | | |
| TPI1 | Triosephosphate isomerase 1 | 7.24E−03 | −1.2237 | −1.3774 | −0.6167 | −1.0726 |
| DEDD | Death effector domain | 8.15E−03 | −1.3712 | −1.1931 | −0.6877 | −1.0840 |
| | containing | | | | | |
| NEDD8 | Neural precursor cell expressed | 4.76E−02 | −1.2564 | −1.0367 | −0.9592 | −1.0841 |
| CSE1L | CSE1 chromosome segregation | 3.78E−02 | −1.3032 | −1.2387 | −0.7256 | −1.0892 |
| | 1-like (yeast) | | | | | |
| APP | Amyloid beta (A4) precursor | 4.47E−02 | −1.2020 | −1.2149 | −0.8969 | −1.1046 |
| | protein (peptidase nexin-II | | | | | |
| NAP1L1 | Nucleosome assembly | 4.53E−02 | −1.5774 | −1.1346 | −0.6020 | −1.1047 |
| | protein 1-like 1 | | | | | |
| POLR2A | Polymerase (RNA) II | 2.26E−02 | −1.2351 | −1.2968 | −0.7919 | −1.1079 |
| | (DNA directed) polypeptide A | | | | | |
| CEP170L | Centrosomal protein | 1.98E−02 | −1.2005 | −1.2684 | −0.8709 | −1.1132 |
| | 170 kDa-like | | | | | |
| MFI2 | Antigen p97 (melanoma | 2.05E−02 | −1.0433 | −1.3673 | −0.9407 | −1.1171 |
| | associated) identified by | | | | | |
| | monoclonal antibodies | | | | | |
| | 133.2 and 96.5 | | | | | |
| DKFZp547C195 | Hypothetical protein | 2.28E−02 | −1.3092 | −1.3446 | −0.7442 | −1.1327 |
| | DKFZp547C195 | | | | | |
| XRCC5 | X-ray repair complementing | 6.76E−03 | −1.4121 | −1.2799 | −0.7349 | −1.1423 |
| | defective repair in Chinese | | | | | |
| | hamster cells 5 (double- | | | | | |
| | strand-break rejoining; Ku | | | | | |
| | autoantigen | | | | | |
| RY1 | Putative nucleic acid | 3.98E−02 | −1.4147 | −1.1024 | −0.9167 | −1.1446 |
| | binding protein RY-1 | | | | | |
| YWHAZ | Tyrosine 3- | 1.81E−02 | −1.0370 | −1.1326 | −1.2976 | −1.1557 |
| | monooxygenase/tryptophan | | | | | |
| | 5-monooxygenase activation | | | | | |
| | protein | | | | | |
| UBC | Ubiquitin C | 4.03E−02 | −1.4084 | −1.3554 | −0.7117 | −1.1585 |
| YWHAZ | Tyrosine 3- | 4.41E−02 | −1.2832 | −1.2893 | −0.9055 | −1.1593 |
| | monooxygenase/tryptophan | | | | | |
| | 5-monooxygenase activation | | | | | |
| | protein | | | | | |
| BTBD14B | BTB (POZ) domain | 2.46E−02 | −1.0817 | −1.4892 | −0.9101 | −1.1603 |
| | containing 14B | | | | | |
| MRPL47 | Mitochondrial ribosomal | 2.09E−02 | −1.4589 | −1.4617 | −0.6253 | −1.1820 |
| | protein L47 | | | | | |
| SMAP1L | Stromal membrane- | 3.55E−02 | −1.4296 | −1.0051 | −1.1201 | −1.1849 |
| | associated protein 1-like | | | | | |
| GOLT1B | Golgi transport 1 homolog | 1.16E−02 | −1.3607 | −1.2870 | −0.9161 | −1.1879 |
| | B (S. cerevisiae) | | | | | |
| TP53BP2 | Tumor protein p53 binding | 1.65E−02 | −1.1983 | −1.4931 | −0.8981 | −1.1965 |
| | protein | | | | | |
| ZNF532 | Zinc finger protein 532 | 2.52E−02 | −1.2384 | −1.2732 | −1.0835 | −1.1984 |
| IARS2 | Isoleucine-tRNA synthetase 2 | 4.91E−02 | −1.4017 | −1.4132 | −0.7866 | −1.2005 |
| RAB10 | RAB10 | 1.24E−02 | −1.3733 | −1.4922 | −0.7861 | −1.2172 |
| YWHAZ | Tyrosine 3- | 3.62E−02 | −1.3707 | −1.3034 | −0.9851 | −1.2197 |
| | monooxygenase/tryptophan | | | | | |
| | 5-monooxygenase activation | | | | | |
| | protein | | | | | |
| RTN4 | Reticulon 4 | 1.07E−02 | −1.5276 | −1.5286 | −0.6137 | −1.2233 |
| APP | Amyloid beta (A4) | 3.40E−02 | −1.5213 | −1.4772 | −0.6731 | −1.2239 |
| | precursor protein (peptidase | | | | | |
| | nexin-II | | | | | |
| SKP1A | S-phase kinase-associated | 2.36E−02 | −1.3285 | −1.3223 | −1.0324 | −1.2278 |
| | protein 1A (p19A) | | | | | |
| PPP1CB | Protein phosphatase 1 | 2.06E−02 | −1.5162 | −1.5934 | −0.6015 | −1.2370 |
| DPF2 | D4 | 1.96E−02 | −1.5602 | −1.5153 | −0.6634 | −1.2463 |
| YWHAZ | Tyrosine 3- | 3.11E−02 | −1.4722 | −1.4013 | −0.8670 | −1.2468 |
| | monooxygenase/tryptophan | | | | | |
| | 5-monooxygenase activation | | | | | |
| | protein | | | | | |
| HP | Haptoglobin | 4.27E−02 | −1.4075 | −1.3329 | −1.0232 | −1.2545 |
| GNA13 | Guanine nucleotide binding | 1.24E−02 | −1.4513 | −1.6015 | −0.7337 | −1.2622 |
| | protein (G protein) | | | | | |
| YWHAZ | Tyrosine 3- | 3.26E−02 | −1.5251 | −1.3519 | −0.9214 | −1.2662 |
| | monooxygenase/tryptophan | | | | | |
| | 5-monooxygenase activation | | | | | |
| | protein | | | | | |
| PARP10 | Poly (ADP-ribose) | 4.84E−02 | −1.6160 | −1.5452 | −0.6725 | −1.2779 |
| | polymerase family | | | | | |
| UBC | Ubiquitin C | 8.05E−03 | −1.5689 | −1.6907 | −0.6095 | −1.2897 |
| RB1 | Retinoblastoma 1 (including | 3.81E−02 | −1.5644 | −1.5840 | −0.7211 | −1.2898 |
| | osteosarcoma) | | | | | |
| NDUFS2 | NADH dehydrogenase | 4.71E−02 | −1.5603 | −1.4593 | −0.8517 | −1.2904 |
| | (ubiquinone) Fe—S protein 2 | | | | | |
| UBC | Ubiquitin C | 4.36E−02 | −1.7413 | −1.4803 | −0.6581 | −1.2932 |
| RPL15 | Ribosomal protein L15 | 4.76E−02 | −1.6086 | −1.5503 | −0.7237 | −1.2942 |
| MLLT11 | Myeloid/lymphoid or | 1.40E−02 | −1.4989 | −1.4863 | −0.9230 | −1.3027 |
| | mixed-lineage leukemia | | | | | |
| | (trithorax homolog | | | | | |
| SPON2 | Spondin 2 | 1.24E−02 | −1.5816 | −1.3046 | −1.0280 | −1.3047 |
| RBM13 | RNA binding motif protein | 2.89E−02 | −1.6818 | −1.5359 | −0.7186 | −1.3121 |
| | 13 | | | | | |
| CLDND2 | Claudin domain containing 2 | 1.30E−02 | −1.5352 | −1.6015 | −0.8384 | −1.3250 |
| PPM1G | Protein phosphatase 1G | 4.74E−02 | −0.9832 | −1.8851 | −1.1180 | −1.3288 |
| | (formerly 2C) | | | | | |
| TWIST1 | Twist homolog 1 | 3.13E−03 | −1.6394 | −1.7669 | −0.6110 | −1.3391 |
| | (acrocephalosyndactyly 3; | | | | | |
| | Saethre-Chotzen syndrome) | | | | | |
| | (Drosophila) | | | | | |
| SLC25A5 | Solute carrier family 25 | 3.75E−02 | −1.6256 | −1.3789 | −1.0131 | −1.3392 |
| | (mitochondrial carrier; | | | | | |
| | adenine nucleotide | | | | | |
| | translocator) | | | | | |
| UBC | Ubiquitin C | 4.23E−02 | −1.7286 | −1.5598 | −0.7344 | −1.3409 |
| GNB2 | Guanine nucleotide binding | 2.31E−02 | −1.6348 | −1.6308 | −0.7601 | −1.3419 |
| | protein (G protein) | | | | | |
| FHL1 | Four and a half LIM | 2.88E−02 | −1.7222 | −1.7108 | −0.6042 | −1.3457 |
| | domains 1 | | | | | |
| UBC | Ubiquitin C | 1.08E−03 | −1.5009 | −1.6030 | −0.9407 | −1.3482 |
| TPM3 | Tropomyosin 3 | 9.06E−03 | −1.4659 | −1.6581 | −0.9356 | −1.3532 |
| HCLS1 | Hematopoietic cell-specific | 2.27E−02 | −0.9264 | −1.0866 | −2.0569 | −1.3566 |
| | Lyn substrate 1 | | | | | |
| UQCRH | Ubiquinol-cytochrome c | 2.63E−02 | −1.1367 | −1.3132 | −1.6853 | −1.3784 |
| | reductase hinge protein | | | | | |
| CCT5 | Chaperonin containing | 2.07E−02 | −1.6909 | −1.7426 | −0.7185 | −1.3840 |
| | TCP1 | | | | | |
| HNRPA2B1 | Heterogeneous nuclear | 3.45E−02 | −1.3362 | −1.4551 | −1.3710 | −1.3874 |
| | ribonucleoprotein A2/B1 | | | | | |
| UBC | Ubiquitin C | 3.11E−02 | −1.6931 | −1.7166 | −0.7601 | −1.3899 |
| BASP1 | Brain abundant | 4.28E−02 | −1.2112 | −1.1801 | −1.7927 | −1.3947 |
| ZMYM2 | Zinc finger protein 198 | 1.00E−02 | −1.8223 | −1.7286 | −0.6440 | −1.3983 |
| ZNF486 | Zinc finger protein 486 | 3.17E−02 | −1.6909 | −1.5791 | −0.9257 | −1.3986 |
| RPL13A | Ribosomal protein L13a | 1.74E−02 | −1.4775 | −1.8345 | −0.8960 | −1.4027 |
| INPPL1 | Inositol polyphosphate | 2.99E−02 | −1.5612 | −1.5684 | −1.0966 | −1.4088 |
| | phosphatase-like 1 | | | | | |
| AOF2 | Amine oxidase (flavin | 2.60E−02 | −1.8936 | −1.7021 | −0.6658 | −1.4205 |
| | containing) domain 2 | | | | | |
| BACE1 | Beta-site APP-cleaving | 4.20E−02 | −1.6356 | −1.4602 | −1.1734 | −1.4231 |
| | enzyme 1 | | | | | |
| GDI2 | GDP dissociation inhibitor 2 | 2.00E−02 | −1.7437 | −1.8670 | −0.6770 | −1.4292 |
| 2-Sep | Septin 2 | 4.22E−02 | −1.6532 | −1.4372 | −1.2036 | −1.4313 |
| CCNG1 | Cyclin G1 | 6.04E−03 | −1.4167 | −1.4652 | −1.4230 | −1.4350 |
| UBC | Ubiquitin C | 2.31E−02 | −1.7875 | −1.7704 | −0.7581 | −1.4386 |
| COX7A2L | Cytochrome c oxidase | 2.79E−02 | −1.6347 | −1.5912 | −1.0918 | −1.4392 |
| | subunit VIIa polypeptide 2 | | | | | |
| | like | | | | | |
| UBC | Ubiquitin C | 1.92E−02 | −1.7052 | −1.7132 | −0.9000 | −1.4395 |
| IMPDH1 | IMP (inosine | 4.19E−02 | −1.1749 | −2.1767 | −1.0024 | −1.4513 |
| | monophosphate) | | | | | |
| | dehydrogenase 1 | | | | | |
| HIST1H2BG | Histone 1 | 3.16E−02 | −2.3323 | −1.3773 | −0.6631 | −1.4575 |
| IARS | Isoleucine-tRNA synthetase | 2.70E−02 | −1.7778 | −1.8215 | −0.8306 | −1.4767 |
| YWHAZ | Tyrosine 3- | 2.32E−02 | −1.7392 | −1.7617 | −0.9727 | −1.4912 |
| | monooxygenase/tryptophan | | | | | |
| | 5-monooxygenase activation | | | | | |
| | protein | | | | | |
| YWHAZ | Tyrosine 3- | 1.46E−02 | −1.6655 | −1.6670 | −1.1452 | −1.4926 |
| | monooxygenase/tryptophan | | | | | |
| | 5-monooxygenase activation | | | | | |
| | protein | | | | | |
| FTH1 | Ferritin | 2.50E−02 | −1.6160 | −1.5550 | −1.3076 | −1.4929 |
| UBC | Ubiquitin C | 2.38E−02 | −1.9110 | −1.9578 | −0.6428 | −1.5038 |
| SFRS10 | Splicing factor | 3.05E−02 | −1.9200 | −1.7444 | −0.8730 | −1.5125 |
| GNL1 | Guanine nucleotide binding | 2.47E−02 | −1.7108 | −1.7469 | −1.1638 | −1.5405 |
| | protein-like 1 | | | | | |
| GTF2A2 | General transcription factor | 1.77E−02 | −1.6295 | −2.3775 | −0.6154 | −1.5408 |
| | IIA | | | | | |
| CCNG1 | Cyclin G1 | 1.92E−02 | −1.9686 | −1.8486 | −0.8159 | −1.5444 |
| HSP90AB1 | Heat shock protein 90 kDa | 3.21E−02 | −1.9063 | −1.7707 | −0.9668 | −1.5479 |
| | alpha (cytosolic) | | | | | |
| TTC25 | Tetratricopeptide repeat | 3.95E−02 | −1.2236 | −1.2854 | −2.1381 | −1.5490 |
| | domain 25 | | | | | |
| ZNF552 | Zinc finger protein 552 | 1.51E−02 | −1.9852 | −1.9950 | −0.6821 | −1.5541 |
| UBC | Ubiquitin C | 1.34E−02 | −2.1019 | −1.9528 | −0.6143 | −1.5563 |
| STMN1 | Stathmin 1/oncoprotein 18 | 2.44E−02 | −1.8871 | −1.8108 | −1.0351 | −1.5777 |
| RPL13A | Ribosomal protein L13a | 1.43E−02 | −2.0024 | −2.1014 | −0.6294 | −1.5778 |
| UBE2Z | Ubiquitin-conjugating | 4.85E−02 | −1.7216 | −2.3116 | −0.7118 | −1.5817 |
| | enzyme E2Z (putative) | | | | | |
| GNG13 | Guanine nucleotide binding | 5.34E−03 | −2.0691 | −2.0669 | −0.6207 | −1.5856 |
| | protein (G protein) | | | | | |
| FASTK | Fas-activated | 2.32E−02 | −1.8197 | −1.7844 | −1.1751 | −1.5931 |
| | serine/threonine kinase | | | | | |
| NCF1 | Neutrophil cytosolic factor 1 | 2.53E−02 | −1.6495 | −1.5468 | −1.5992 | −1.5985 |
| RB1 | Retinoblastoma 1 (including | 3.10E−02 | −1.5469 | −1.5264 | −1.7257 | −1.5997 |
| | osteosarcoma) | | | | | |
| AQP7P1 | Aquaporin 7 pseudogene 1 | 3.70E−02 | −1.5607 | −1.4481 | −1.8260 | −1.6116 |
| TFG | TRK-fused gene | 1.72E−02 | −1.9243 | −1.8209 | −1.0934 | −1.6129 |
| NOL11 | Nucleolar protein 11 | 2.88E−02 | −1.4716 | −2.2097 | −1.1689 | −1.6168 |
| MGC46336 | Hypothetical protein | 2.38E−02 | −2.0394 | −1.9741 | −0.8383 | −1.6173 |
| | MGC46336 | | | | | |
| PCTK3 | PCTAIRE protein kinase 3 | 2.88E−02 | −1.5533 | −1.5669 | −1.7442 | −1.6215 |
| MTHFD2 | Methylenetetrahydrofolate | 1.45E−02 | −2.0912 | −2.1265 | −0.6489 | −1.6222 |
| | dehydrogenase (NADP+ | | | | | |
| | dependent) 2 | | | | | |
| HIST1H2BN | Histone 1 | 2.03E−02 | −2.0102 | −1.8981 | −0.9761 | −1.6281 |
| PPIA | Peptidylprolyl isomerase A | 4.09E−03 | −1.2029 | −1.1585 | −2.5357 | −1.6323 |
| | (cyclophilin A) | | | | | |
| GAPDH | Glyceraldehyde-3- | 2.60E−02 | −2.1595 | −2.1137 | −0.6837 | −1.6523 |
| | phosphate dehydrogenase | | | | | |
| E2F3 | E2F transcription factor 3 | 1.28E−02 | −2.1246 | −2.1763 | −0.6840 | −1.6617 |
| RPL13A | Ribosomal protein L13a | 1.40E−02 | −2.2136 | −2.1208 | −0.6950 | −1.6765 |
| RPS27A | Ribosomal protein S27a | 1.10E−02 | −1.7599 | −1.7528 | −1.5771 | −1.6966 |
| ACN9 | ACN9 homolog (S. cerevisiae) | 3.19E−02 | −1.6725 | −2.1626 | −1.2833 | −1.7061 |
| RPL30 | Ribosomal protein L30 | 9.69E−03 | −2.2892 | −2.1662 | −0.6640 | −1.7065 |
| CHTF18 | CTF18 | 1.93E−02 | −2.0431 | −2.1087 | −0.9860 | −1.7126 |
| YWHAZ | Tyrosine 3- | 2.81E−02 | −2.2482 | −2.1431 | −0.7763 | −1.7225 |
| | monooxygenase/tryptophan | | | | | |
| | 5-monooxygenase activation | | | | | |
| | protein | | | | | |
| LDHB | Lactate dehydrogenase B | 4.77E−02 | −2.2976 | −2.1288 | −0.7496 | −1.7253 |
| RPS27 | Ribosomal protein S27 | 2.44E−02 | −2.2364 | −2.2011 | −0.7419 | −1.7265 |
| | (metallopanstimulin 1) | | | | | |
| GMIP | GEM interacting protein | 2.32E−02 | −2.1179 | −2.0560 | −1.0194 | −1.7311 |
| PRR13 | Proline rich 13 | 3.04E−02 | −1.6928 | −1.6709 | −1.8315 | −1.7318 |
| GAPDH | Glyceraldehyde-3- | 1.64E−02 | −1.9147 | −2.0948 | −1.1906 | −1.7334 |
| | phosphate dehydrogenase | | | | | |
| YBX1 | Y box binding protein 1 | 1.02E−03 | −2.1910 | −2.2579 | −0.7545 | −1.7345 |
| WDR32 | WD repeat domain 32 | 2.59E−02 | −2.1782 | −1.6270 | −1.4254 | −1.7435 |
| TUBB2C | Tubulin | 2.45E−02 | −2.3272 | −2.2857 | −0.6236 | −1.7455 |
| C3orf58 | Chromosome 3 open | 2.29E−02 | −2.1665 | −2.4343 | −0.6458 | −1.7489 |
| | reading frame 58 | | | | | |
| RPL13A | Ribosomal protein L13a | 1.54E−02 | −2.3031 | −2.3129 | −0.6466 | −1.7542 |
| PTEN | Phosphatase and tensin | 1.98E−02 | −1.7294 | −1.6180 | −1.9221 | −1.7565 |
| | homolog (mutated in | | | | | |
| | multiple advanced cancers | | | | | |
| | 1) | | | | | |
| PLA2G2F | Phospholipase A2 | 7.93E−03 | −1.5063 | −1.8233 | −1.9414 | −1.7570 |
| UBC | Ubiquitin C | 1.65E−02 | −2.3447 | −2.1781 | −0.7490 | −1.7573 |
| CCT4 | Chaperonin containing | 2.21E−02 | −1.5954 | −1.7016 | −1.9800 | −1.7590 |
| | TCP1 | | | | | |
| UQCRH | Ubiquinol-cytochrome c | 3.78E−02 | −1.8738 | −1.6913 | −1.7263 | −1.7638 |
| | reductase hinge protein | | | | | |
| LOC92017 | Similar to RIKEN cDNA | 2.15E−02 | −2.6519 | −2.0051 | −0.6357 | −1.7643 |
| | 4933437K13 | | | | | |
| GAPDH | Glyceraldehyde-3- | 3.08E−02 | −2.3307 | −2.1374 | −0.8495 | −1.7725 |
| | phosphate dehydrogenase | | | | | |
| HSF1 | Heat shock transcription | 4.31E−02 | −1.3472 | −1.2958 | −2.7022 | −1.7817 |
| | factor 1 | | | | | |
| HDAC2 | Histone deacetylase 2 | 2.72E−02 | −2.4022 | −2.0698 | −0.8824 | −1.7848 |
| RPL13A | Ribosomal protein L13a | 4.08E−03 | −2.2791 | −2.3609 | −0.8455 | −1.8285 |
| CLPP | ClpP caseinolytic peptidase | 2.61E−02 | −1.3107 | −1.3858 | −2.7939 | −1.8301 |
| C7orf26 | Chromosome 7 open | 3.34E−04 | −2.4606 | −2.4844 | −0.7023 | −1.8824 |
| | reading frame 26 | | | | | |
| GABRB3 | Gamma-aminobutyric acid | 8.48E−03 | −2.1811 | −2.6266 | −0.8688 | −1.8922 |
| | (GABA) A receptor | | | | | |
| ASCC2 | Activating signal | 3.58E−02 | −1.0564 | −1.1024 | −3.6044 | −1.9211 |
| | cointegrator 1 complex | | | | | |
| | subunit 2 | | | | | |
| PRSS1 | Protease | 4.20E−02 | −1.2142 | −1.6243 | −2.9886 | −1.9424 |
| RASSF1 | Ras association | 3.31E−02 | −1.6039 | −1.5479 | −2.6784 | −1.9434 |
| | (RalGDS/AF-6) domain | | | | | |
| | family 1 | | | | | |
| YWHAG | Tyrosine 3- | 9.04E−03 | −2.4647 | −2.3762 | −1.0010 | −1.9473 |
| | monooxygenase/tryptophan | | | | | |
| | 5-monooxygenase activation | | | | | |
| | protein | | | | | |
| HNRPR | Heterogeneous nuclear | 4.98E−02 | −2.0300 | −1.7777 | −2.1297 | −1.9792 |
| | ribonucleoprotein R | | | | | |
| UBE2N | Ubiquitin-conjugating | 2.42E−02 | −2.5855 | −2.4489 | −0.9345 | −1.9896 |
| | enzyme E2N (UBC13 | | | | | |
| | homolog | | | | | |
| BAIAP2L2 | BAI1-associated protein 2- | 2.43E−02 | −1.7850 | −1.7419 | −2.4439 | −1.9903 |
| | like 2 | | | | | |
| C10orf6 | Chromosome 10 open | 4.69E−02 | −3.4495 | −1.8585 | −0.8266 | −2.0449 |
| | reading frame 6 | | | | | |
| RPS8 | Ribosomal protein S8 | 2.41E−02 | −2.4310 | −2.4426 | −1.2951 | −2.0562 |
| SETD3 | SET domain containing 3 | 4.76E−02 | −3.3682 | −2.1626 | −0.6547 | −2.0618 |
| CDK5RAP1 | CDK5 regulatory subunit | 4.08E−02 | −3.1665 | −2.1120 | −0.9712 | −2.0832 |
| | associated protein 1 | | | | | |
| LRRC38 | Leucine rich repeat | 1.83E−02 | −0.9960 | −1.1877 | −4.1113 | −2.0983 |
| | containing 38 | | | | | |
| FGF14 | Fibroblast growth factor 14 | 1.79E−03 | −1.9563 | −2.1432 | −2.2633 | −2.1210 |
| HSPA4 | Heat shock 70 kDa protein 4 | 3.77E−02 | −1.7919 | −1.5779 | −3.0719 | −2.1472 |
| TEAD1 | TEA domain family | 2.13E−02 | −3.2820 | −2.4746 | −0.7251 | −2.1606 |
| | member 1 (SV40 | | | | | |
| | transcriptional enhancer | | | | | |
| | factor) | | | | | |
| DCAMKL3 | Doublecortin and CaM | 5.06E−03 | −1.5032 | −1.4569 | −3.5264 | −2.1622 |
| | kinase-like 3 | | | | | |
| HCAP-G | Chromosome condensation | 2.20E−02 | −1.8816 | −1.7541 | −2.9212 | −2.1857 |
| | protein G | | | | | |
| GALK2 | Galactokinase 2 | 1.74E−02 | −1.8034 | −1.7083 | −3.1113 | −2.2077 |
| ALKBH8 | AlkB | 1.32E−02 | −3.0250 | −2.8935 | −0.7096 | −2.2094 |
| U2AF1L1 | U2 small nuclear RNA | 3.15E−02 | −2.1319 | −2.0567 | −2.5454 | −2.2447 |
| | auxillary factor 1-like 1 | | | | | |
| RCC1 | Regulator of chromosome | 2.28E−02 | −1.9147 | −1.9087 | −2.9414 | −2.2549 |
| | condensation 1 | | | | | |
| PABPCP2 | Poly(A) binding protein | 2.44E−02 | −2.4210 | −3.5714 | −0.7940 | −2.2621 |
| RORB | RAR-related orphan | 4.33E−03 | −2.9876 | −2.6622 | −1.1428 | −2.2642 |
| | receptor B | | | | | |
| EHBP1 | EH domain binding protein 1 | 1.30E−02 | −2.6859 | −3.4339 | −0.7762 | −2.2987 |
| TNFSF5IP1 | Tumor necrosis factor | 2.85E−04 | −2.9321 | −2.8896 | −1.1944 | −2.3387 |
| | superfamily | | | | | |
| NCKAP1 | NCK-associated protein 1 | 1.03E−03 | −3.0925 | −2.9485 | −1.0329 | −2.3580 |
| SCC-112 | SCC-112 protein | 7.02E−03 | −3.2596 | −2.7965 | −1.0245 | −2.3602 |
| DISC1 | Disrupted in schizophrenia 1 | 2.23E−02 | −2.7536 | −3.2161 | −1.3263 | −2.4320 |
| FRAS1 | Fraser syndrome 1 | 7.35E−03 | −3.0410 | −3.6622 | −0.6369 | −2.4467 |
| RP11-82K18.3 | Kynurenine | 4.10E−02 | −3.5108 | −3.0051 | −1.0254 | −2.5137 |
| | aminotransferase III | | | | | |
| SNX16 | Sorting nexin 16 | 4.52E−02 | −4.2138 | −2.7309 | −0.6029 | −2.5159 |
| MTMR4 | Myotubularin related | 1.86E−02 | −2.7799 | −4.0428 | −0.7256 | −2.5161 |
| | protein 4 | | | | | |
| RUFY3 | RUN and FYVE domain | 6.44E−03 | −3.5450 | −3.0595 | −1.0449 | −2.5498 |
| | containing 3 | | | | | |
| TUBE1 | Tubulin | 2.46E−02 | −4.0145 | −2.9485 | −0.6945 | −2.5525 |
| RNF41 | Ring finger protein 41 | 5.42E−04 | −3.3889 | −3.5900 | −0.7154 | −2.5648 |
| ZNF650 | Zinc finger protein 650 | 4.19E−02 | −2.5111 | −2.4103 | −2.9008 | −2.6074 |
| PTPLAD1 | Protein tyrosine | 3.49E−02 | −3.3024 | −3.4629 | −1.0889 | −2.6181 |
| | phosphatase-like A domain | | | | | |
| | containing 1 | | | | | |
| SMG1 | PI-3-kinase-related kinase | 1.47E−04 | −3.5076 | −3.6266 | −0.7636 | −2.6326 |
| | SMG-1 | | | | | |
| TUBB3 | Tubulin | 4.76E−02 | −2.8608 | −1.8681 | −3.1978 | −2.6422 |
| KLHL12 | Kelch-like 12 (Drosophila) | 1.39E−02 | −2.8743 | −2.3046 | −2.8057 | −2.6616 |
| LOC144486 | Hypothetical protein | 1.30E−02 | −3.7026 | −3.4113 | −0.9092 | −2.6743 |
| | LOC144486 | | | | | |
| TYMS | Thymidylate synthetase | 3.99E−02 | −4.2342 | −3.2115 | −0.6514 | −2.6990 |
| BAX | BCL2-associated X protein | 1.10E−02 | −3.3257 | −4.1626 | −0.6184 | −2.7022 |
| SFRS15 | Splicing factor | 2.44E−02 | −3.6347 | −3.7600 | −0.7271 | −2.7073 |
| THSD1P | Thrombospondin | 1.53E−02 | −2.2065 | −2.3813 | −3.5917 | −2.7265 |
| ARF4 | ADP-ribosylation factor 4 | 1.66E−02 | −3.9601 | −3.0860 | −1.1560 | −2.7340 |
| TTN | Titin | 7.54E−05 | −3.7354 | −3.8282 | −0.7170 | −2.7602 |
| FLT3 | Fms-related tyrosine kinase 3 | 4.11E−02 | −2.3385 | −4.1371 | −1.8379 | −2.7711 |
| SHPRH | SNF2 histone linker PHD | 2.11E−02 | −2.8743 | −3.2394 | −2.2014 | −2.7717 |
| | RING helicase | | | | | |
| MINA | MYC induced nuclear | 2.62E−02 | −3.1904 | −4.5043 | −0.6722 | −2.7889 |
| | antigen | | | | | |
| JTB | Jumping translocation | 3.83E−02 | −3.9524 | −3.4132 | −1.1453 | −2.8370 |
| | breakpoint | | | | | |
| MRPS27 | Mitochondrial ribosomal | 4.42E−02 | −3.3889 | −3.4317 | −1.7065 | −2.8424 |
| | protein S27 | | | | | |
| TMEM49 | Transmembrane protein 49 | 3.98E−05 | −3.7674 | −3.7965 | −0.9944 | −2.8528 |
| IVNS1ABP | Influenza virus NS1A | 1.71E−02 | −3.0925 | −4.0860 | −1.5093 | −2.8959 |
| | binding protein | | | | | |
| MDM2 | Mdm2 | 4.85E−02 | −4.1544 | −3.9042 | −0.6314 | −2.8967 |
| TBC1D4 | TBC1 domain family | 1.80E−02 | −4.4884 | −3.4544 | −0.7545 | −2.8991 |
| ELAVL3 | ELAV (embryonic lethal | 1.14E−02 | −2.6396 | −2.6420 | −3.5397 | −2.9404 |
| TMEM30A | Transmembrane protein | 2.55E−02 | −3.0544 | −4.8203 | −1.0536 | −2.9761 |
| | 30A | | | | | |
| DYNC2LI1 | Dynein | 4.26E−02 | −3.1904 | −4.9771 | −0.7785 | −2.9820 |
| NXT2 | Nuclear transport factor 2- | 3.49E−02 | −3.0032 | −5.0728 | −1.0566 | −3.0442 |
| | like export factor 2 | | | | | |
| PIP5K1C | Phosphatidylinositol-4- | 3.44E−02 | −4.6859 | −3.3193 | −1.1339 | −3.0464 |
| | phosphate 5-kinase | | | | | |
| HIAT1 | Hippocampus abundant | 3.15E−02 | −4.8817 | −3.1202 | −1.1461 | −3.0493 |
| | transcript 1 | | | | | |
| LAYN | Layilin | 4.88E−02 | −4.9461 | −3.1375 | −1.0784 | −3.0540 |
| C21orf100 | Chromosome 21 open | 4.79E−02 | −3.9321 | −2.5141 | −2.8590 | −3.1017 |
| | reading frame 100 | | | | | |
| TBC1D7 | TBC1 domain family | 6.79E−04 | −4.2254 | −4.3730 | −0.8293 | −3.1426 |
| ALG6 | Asparagine-linked | 3.26E−02 | −5.1904 | −3.6084 | −0.7504 | −3.1831 |
| | glycosylation 6 homolog (S. cerevisiae | | | | | |
| PDGFRA | Platelet-derived growth | 6.11E−03 | −3.8890 | −4.5992 | −1.1636 | −3.2173 |
| | factor receptor | | | | | |
| IGF2R | Insulin-like growth factor 2 | 1.93E−02 | −4.9035 | −3.7309 | −1.2226 | −3.2856 |
| | receptor | | | | | |
| LRP1 | Low density lipoprotein- | 3.34E−03 | −4.4022 | −4.6865 | −0.8832 | −3.3240 |
| | related protein 1 (alpha-2- | | | | | |
| | macroglobulin receptor) | | | | | |
| FAM35A | Family With sequence | 3.73E−03 | −4.2709 | −4.8745 | −0.9458 | −3.3637 |
| | similarity 35 | | | | | |
| GATA1 | GATA binding protein 1 | 4.79E−02 | −3.6532 | −3.3909 | −3.1865 | −3.4102 |
| | (globin transcription factor | | | | | |
| | 1) | | | | | |
| CBR4 | Carbonyl reductase 4 | 4.66E−02 | −3.7026 | −5.8933 | −0.7333 | −3.4431 |
| TXNDC13 | Thioredoxin domain | 4.10E−04 | −4.8219 | −4.6797 | −0.8778 | −3.4598 |
| | containing 13 | | | | | |
| TIGD4 | Tigger transposable element | 9.88E−03 | −3.3953 | −4.3485 | −2.6887 | −3.4775 |
| | derived 4 | | | | | |
| PRC1 | Protein regulator of | 8.16E−03 | −5.2196 | −4.3921 | −0.9417 | −3.5178 |
| | cytokinesis 1 | | | | | |
| NRG1 | Neuregulin 1 | 4.36E−02 | −5.3311 | −3.4746 | −2.1579 | −3.6545 |
| TJP1 | Tight junction protein 1 | 2.99E−03 | −4.8047 | −5.0270 | −1.2888 | −3.7068 |
| | (zona occludens 1) | | | | | |
| LARP6 | La ribonucleoprotein | 1.27E−02 | −5.7272 | −4.5992 | −1.4613 | −3.9292 |
| | domain family | | | | | |
| OS9 | Amplified in osteosarcoma | 1.91E−02 | −2.9016 | −4.1873 | −5.0030 | −4.0306 |
| SSX6 | Synovial sarcoma | 5.13E−03 | −4.7507 | −5.1593 | −3.1814 | −4.3638 |

All references cited in this specification are herein incorporated by reference as though each reference was specifically and individually indicated to be incorporated by reference. The citation of any reference is for its disclosure prior to the filing date and should not be construed as an admission that the present invention is not entitled to antedate such reference by virtue of prior invention.

It will be understood that each of the elements described above, or two or more together may also find a useful application in other types of methods differing from the type described above. Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention set forth in the appended claims. The foregoing embodiments are presented by way of example only.

The invention claimed is:

1. A method of culturing neurons, comprising:
a) isolating transformed neuronal cells; and
b) culturing said transformed neuronal cells in 3-D culture, said 3-D culture comprising a rotating wall vessel containing said transformed neuronal cells, culture media, and a cell culture matrix, wherein said rotating wall vessel gravity is balanced by oppositely directed physical forces, and so generating 3-D cultured cells;
whereby the 3-D cultured cells adopt a 3-D phenotype, and wherein said 3-D phenotype persists for up to 5 days after said 3-D cultured cells are transferred to 2-D culture.

2. The method of claim 1, wherein said 3-D phenotype comprises decreased N-myc expression.

3. The method of claim 1, wherein said 3-D phenotype comprises decreased HuD expression.

4. The method of claim 1, wherein said 3-D phenotype comprises decreased Bcl-2 expression.

5. The method of claim 1, wherein said 3-D phenotype comprises increased Bax expression.

6. The method of claim 1, wherein said 3-D phenotype comprises increased Bak expression.

7. The method of claim 1, wherein said 3-D phenotype comprises increased susceptibility to apoptosis.

8. The method of claim 1, wherein said 3-D phenotype comprises increased neurite outgrowth.

9. The method of claim 1, wherein said 3-D phenotype comprises decreased doubling rate.

10. A transformed neuronal cell with 3-D phenotype, wherein said 3-D phenotype comprises: reduced doubling rate; increased susceptibility to apoptosis; and increased neurite formation.

11. The cell of claim 10, wherein said 3-D phenotype further comprises: reduced N-myc expression; reduced HuD expression; reduced Bcl-2 expression; increased Bax expression; and increased Bak expression.

12. The cell of claim 10, wherein said 3-D phenotype persists for up to 5 days after said cell is transferred to 2-D culture.

13. The cell of claim 12 wherein said transformed neuronal cell is an SH-SY5Y cell or a PC12 cell.

14. The cell of claim 11, wherein said 3-D phenotype persists for up to 5 days after said cell is transferred to 2-D culture.

15. The cell of claim 14 wherein said transformed neuronal cell is an SH-SY5Y cell or a PC12 cell.
